(19) United States
(12) Patent Application Publication
(10) Pub. No.: US 2013/0217647 A1
(43) Pub. Date: Aug. 22, 2013

(54) Biomarkers for Prostate Cancer and Methods Using the Same

(71) Applicants: Jeffrey R. Shuster, Chapel Hill, NC (US); Matthew W. Mitchell, Durham, NC (US); Jonathan E. McDunn, Cary, NC (US); Bruce Neri, Cary, NC (US)

(72) Inventors: Jeffrey R. Shuster, Chapel Hill, NC (US); Matthew W. Mitchell, Durham, NC (US); Jonathan E. McDunn, Cary, NC (US); Bruce Neri, Cary, NC (US)

(73) Assignee: METABOLON, INC., Durham, NC (US)

(21) Appl. No.: 13/812,183

(22) PCT Filed: Jul. 27, 2011
(86) PCT No.: PCT/US11/45514

§ 371 Date: May 1, 2013

Related U.S. Application Data
(60) Provisional application No. 61/368,434, filed on Jul. 28, 2010.

(51) Int. Cl. G01N 33/574; A61K 31/7072; A61K 31/685

(52) U.S. Cl. CPC G01N 33/57434; A61K 31/685; A61K 31/7072
USPC 514/51; 436/501; 435/7.92; 435/7.1; 514/114

(57) ABSTRACT

Biomarkers (and suites of biomarkers) relating to prostate cancer are provided, as well as methods for using such biomarkers (ans suites thereof), including early prediction of prostate cancer, disease grading, target identification/validation, and monitoring of drug efficacy.

This application claims the benefit of U.S. Provisional Patent Application No. 61/368,434, filed Jul. 28, 2010, the entire contents of which are hereby incorporated herein by reference.

### FIELD

The invention generally relates to biomarkers for prostate cancer and methods based on the same biomarkers.

### BACKGROUND

Prostate cancer is the leading cause of male cancer-related deaths and afflicts one out of nine men over the age of 65. The American Cancer Society estimates that over 200,000 American men will be diagnosed with prostate cancer and over 30,000 will die this year. While effective surgical and radiation treatments exist for localized prostate cancer, metastatic prostate cancer remains essentially incurable and most men diagnosed with metastatic disease will succumb over a period of months to years.

Prostate cancer is detected by either a digital rectal exam (DRE), or by the measurement of levels of prostate specific antigen (PSA), which has an unacceptably high rate of false-positives. The diagnosis of prostate cancer can be confirmed only by a biopsy. Radical prostatectomy, radiation and watchful waiting are generally effective for localized prostate cancer, but it is often difficult to determine which approach to use. Since it is not possible to distinguish between the indolent and more aggressive tumors current therapy takes a very conservative approach.

While imaging, X-rays, computerized tomography scans and further biopsies can help determine if prostate cancer has metastasized, they are not able to differentiate early stages. Understanding the progression of prostate cancer from a localized, early, indolent state, to an aggressive state, and, ultimately, to a metastatic state would allow the proper clinical management of this disease. Furthermore, early-indolent prostate cancer may be progressive or non-progressive toward aggressive forms.

### SUMMARY

In one aspect, the present invention provides a method of diagnosing whether a subject has prostate cancer, comprising analyzing a biological sample from a subject to determine the level(s) of one or more biomarkers for prostate cancer in the sample, where the one or more biomarkers are selected from Tables 1A, 1B, 3A, 3B, 5A, 5B, 7A, 7B, 8, and/or 10 and comparing the level(s) of the one or more biomarkers in the sample to prostate cancer-positive and/or prostate cancer-negative reference levels of the one or more biomarkers in order to diagnose whether the subject has prostate cancer. The one or more biomarkers may be selected from Tables 1A, 1B, 3A, 3B, and 8. When the biological sample is prostate tissue the one or more biomarkers may be selected from Tables 1A, 1B, 3A, 3B, 5A, 5B, 7A, 7B, 8, and/or 10, or may be selected from Tables 1A, 1B, 3A, 3B, 5A, 5B, 7A, 7B, and/or 10. When the biological sample is urine the one or more biomarkers may be selected from Tables 1A, 1B, 3A, 3B, 5A, 5B, 7A, 7B, 8, and/or 10, or may be selected from Table 8. The biological sample may be a DRE urine sample.

In another aspect, the present invention also provides a method of determining whether a subject is predisposed to developing prostate cancer, comprising analyzing a biological sample from a subject to determine the level(s) of one or more biomarkers for prostate cancer in the sample, where the one or more biomarkers are selected from Tables 1A, 1B, 3A, 3B, 5A, 5B, 7A, 7B, 8, and/or 10; and comparing the level(s) of the one or more biomarkers in the sample to prostate cancer-positive and/or prostate cancer-negative reference levels of the one or more biomarkers in order to determine whether the subject is predisposed to developing prostate cancer.

In yet another aspect, the invention provides a method of monitoring progression/regression of prostate cancer in a subject comprising analyzing a first biological sample from a subject to determine the level(s) of one or more biomarkers for prostate cancer in the sample, where the one or more biomarkers are selected from Tables 1A, 1B, 3A, 3B, 5A, 5B, 7A, 7B, 8, and/or 10 and the first sample is obtained from the subject at a first time point; analyzing a second biological sample from a subject to determine the level(s) of the one or more biomarkers, where the second sample is obtained from the subject at a second time point; and comparing the level(s) of one or more biomarkers in the first sample to the level(s) of the one or more biomarkers in the second sample in order to monitor the progression/regression of prostate cancer in the subject.

In another aspect, the present invention provides a method of assessing the efficacy of a composition for treating prostate cancer comprising analyzing, from a subject having prostate cancer and currently or previously being treated with a composition, a biological sample to determine the level(s) of one or more biomarkers for prostate cancer selected from Tables 1A, 1B, 3A, 3B, 5A, 5B, 7A, 7B, 8, and/or 10; and comparing the level(s) of the one or more biomarkers in the sample to (a) levels of the one or more biomarkers in a previously-taken biological sample from the subject, where the previously-taken biological sample was obtained from the subject before being treated with the composition, (b) prostate cancer-positive reference levels of the one or more biomarkers, and/or (c) prostate cancer-negative reference levels of the one or more biomarkers.

In another aspect, the present invention provides a method for assessing the efficacy of a composition in treating prostate cancer, comprising analyzing a first biological sample from a subject to determine the level(s) of one or more biomarkers for prostate cancer selected from Tables 1A, 1B, 3A, 3B, 5A, 5B, 7A, 7B, 8, and/or 10, the first sample obtained from the subject at a first time point; administering the composition to the subject; analyzing a second biological sample from the subject to determine the level(s) of the one or more biomarkers, the second sample obtained from the subject at a second time point after administration of the composition; comparing the level(s) of one or more biomarkers in the first sample to the level(s) of the one or more biomarkers in the second sample in order to assess the efficacy of the composition for treating prostate cancer.

In yet another aspect, the invention provides a method of assessing the relative efficacy of two or more compositions for treating prostate cancer comprising analyzing, from a first subject having prostate cancer and currently or previously being treated with a first composition, a first biological sample to determine the level(s) of one or more biomarkers selected from Tables 1A, 1B, 3A, 3B, 5A, 5B, 7A, 7B, 8, and/or 10; analyzing, from a second subject having prostate cancer and currently or previously being treated with a second composition, a second biological sample to determine the level(s) of the one or more biomarkers; and comparing the level(s) of one or more biomarkers in the first sample to the level(s) of the one or more biomarkers in the second sample in order to assess the relative efficacy of the first and second compositions for treating prostate cancer.

In another aspect, the present invention provides a method for screening a composition for activity in modulating one or more biomarkers of prostate cancer, comprising contacting one or more cells with a composition; analyzing at least a portion of the one or more cells or a biological sample associated with the cells to determine the level(s) of one or more biomarkers of prostate cancer selected from Tables 1A, 1B, 3A, 3B, 5A, 5B, 7A, 7B, 8, and/or 10; and comparing the level(s) of the one or more biomarkers with predetermined standard levels for the biomarkers to determine whether the composition modulated the level(s) of the one or more biomarkers.

In a further aspect, the present invention provides a method for identifying a potential drug target for prostate cancer comprising identifying one or more biochemical pathways associated with one or more biomarkers for prostate cancer selected from Tables 1A, 1B, 3A, 3B, 5A, 5B, 7A, 7B, 8, and/or 10; and identifying a protein affecting at least one of the one or more identified biochemical pathways, the protein being a potential drug target for prostate cancer.

In yet another aspect, the invention provides a method for treating a subject having prostate cancer comprising administering to the subject an effective amount of one or more biomarkers selected from Tables 1A, 1B, 3A, 3B, 5A, 5B, 7A, 7B, 8, and/or 10 that are decreased in prostate cancer. In another aspect, the invention also provides a method of distinguishing low grade (less aggressive) prostate cancer from high grade (high aggressive) prostate cancer in a subject having prostate cancer, comprising analyzing a biological sample from a subject to determine the level(s) of one or more biomarkers for low grade prostate cancer and/or high grade prostate cancer in the sample, where the one or more biomarkers are selected from Tables 1A, 1B, 3A, 3B, 5A, 5B, 7A, 7B, 8, and/or 10 and comparing the level(s) of the one or more biomarkers in the sample to low grade prostate cancer-positive reference levels that distinguish over high grade prostate cancer and/or to high grade prostate cancer-positive reference levels that distinguish over low grade prostate cancer in order to determine whether the subject has low grade or high grade prostate cancer. The one or more biomarkers may be selected from Tables 1A, 1B, 5A, 5B, 7A, 7B, 8 and/or 10. When the biological sample is prostate tissue, the one or more biomarkers may be selected from Tables 1A, 1B, 3A, 3B, 5A, 5B, 7A, 7B, 8, and/or 10; may be selected from Tables 1A, 1B, 3A, 3B, 5A, 5B, 7A, 7B, and/or 10; or may be selected from Table 10. When selected from Table 10, the biomarkers may be selected from putrescine, lactate, 5,6-dihydrouracil, 10-nonadecenoate, NAD+, spermine, N-acetylputrescine, succinylcarnitine, 3-(4-hydroxyphenyl)lactate, 2-palmitoylglycerophosphoethanolamine, spermidine, glycerol-2-phosphate, glycylvaline, and/or phosphoethanolamine; may be selected from putrescine, lactate, 5,6-dihydrouracil, 10-nonadecenoate, NAD+, spermine, and/or N-acetylputrescine; may be selected from putrescine, glycerol-2-phosphate, and/or glycylvaline; may be selected from phosphoethanolamine, putrescine, and/or spermidine; may be selected from succinylcarnitine, 3-(4-hydroxyphenyl)lactate, 2-palmitoylglycerophosphoethanolamine, lactate, and/or spermidine; and/or may be selected from putrescine, lactate, 5,6-dihydrouracil, 10-nonadecenoate, NAD+, spermine, N-acetylputrescine, succinylcarnitine, 3-(4-hydroxyphenyl)lactate, 2-palmitoylglycerophosphoethanolamine, spermidine, glycerol-2-phosphate, glycylvaline, and/or phosphoethanolamine.

### BRIEF DESCRIPTION OF THE DRAWINGS

FIG. 1 provides a recursive partitioning plot based on one example metabolite (adrenate) to distinguish between subjects with high aggressive prostate cancer and low aggressive prostate cancer (Left) and the corresponding receiver operating characteristic (ROC), or ROC curve, graphical plot of the sensitivity, or true positives, vs. (1—specificity), or false positives (Right).

FIG. 2 provides boxplots of representative biomarker metabolites that are correlated in abundance with cancer. The AUCs for the individual biomarker metabolites range from 0.73 to 0.84. The level of the biomarker in the benign (non-cancer) DRE urine sediment samples is presented on the left and the cancer samples is on the right.

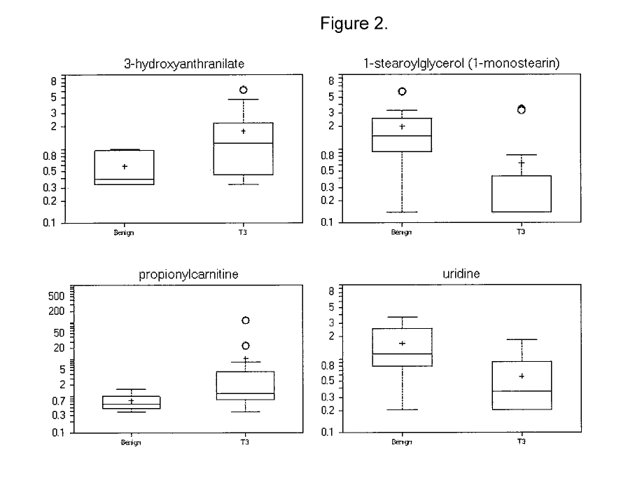

FIG. 3 provides a Receiver Operator Characteristics (ROC) curve for the current state of the art tests for prostate cancer detection, the “Post-DRE PCA 3” (PCA3) test and the “Serum PSA” (PSA) test. The Area Under the Curve (AUC) for the PCA3 test was approximately 0.68 and the AUC for the PSA test was approximately 0.61.

FIG. 4 is a heat map that illustrates the biomarker signatures from DRE urine sediment samples that are associated with prostate cancer. Groups 1 and 2 are biomarker signatures of prostate cancer while Group 3 is a biomarker signature of non-cancer. The cancer biomarker signatures (Group 1 and Group 2) further distinguish subtypes of prostate cancer.

FIG. 5 shows an ROC curve for the Han nomogram described in Example 7.

### DETAILED DESCRIPTION

The present invention relates to biomarkers of prostate cancer, methods for diagnosis of prostate cancer, methods of distinguishing between less aggressive and high aggressive prostate cancer, methods of determining predisposition to prostate cancer, methods of monitoring progression/regression of prostate cancer, methods of assessing efficacy of compositions for treating prostate cancer, methods of screening compositions for activity in modulating biomarkers of prostate cancer, methods of treating prostate cancer, as well as other methods based on biomarkers of prostate cancer. Prior to describing this invention in further detail, however, the following terms will first be defined.

### DEFINITIONS

“Biomarker” means a compound, preferably a metabolite, that is differentially present (i.e., increased or decreased) in a biological sample from a subject or a group of subjects having a first phenotype (e.g., having a disease) as compared to a biological sample from a subject or group of subjects having a second phenotype (e.g., not having the disease). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (i.e., a p-value less than 0.05 and/or a q-value of less than 0.10 as determined using either Welch's T-test or Wilcoxon's rank-sum Test).

The “level” of one or more biomarkers means the absolute or relative amount or concentration of the biomarker in the sample.

“Sample” or “biological sample” means biological material isolated from a subject. The biological sample may contain any biological material suitable for detecting the desired biomarkers, and may comprise cellular and/or non-cellular material from the subject. The sample can be isolated from any suitable biological tissue or fluid such as, for example, prostate tissue, blood, blood plasma, urine, or cerebral spinal fluid (CSF).

“Subject” means any animal, but is preferably a mammal, such as, for example, a human, monkey, mouse, or rabbit.

A “reference level” of a biomarker means a level of the biomarker that is indicative of a particular disease state, phenotype, or lack thereof, as well as combinations of disease states, phenotypes, or lack thereof A “positive” reference level of a biomarker means a level that is indicative of a particular disease state or phenotype. A “negative” reference level of a biomarker means a level that is indicative of a lack of a particular disease state or phenotype. For example, a “prostate cancer-positive reference level” of a biomarker means a level of a biomarker that is indicative of a positive diagnosis of prostate cancer in a subject, and a “prostate cancer-negative reference level” of a biomarker means a level of a biomarker that is indicative of a negative diagnosis of prostate cancer in a subject. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched so that comparisons may be made between biomarker levels in samples from subjects of a certain age and reference levels for a particular disease state, phenotype, or lack thereof in a certain age group). Such reference levels may also be tailored to specific techniques that are used to measure levels of biomarkers in biological samples (e.g., LC-MS, GC-MS, etc.), where the levels of biomarkers may differ based on the specific technique that is used.

“Non-biomarker compound” means a compound that is not differentially present in a biological sample from a subject or a group of subjects having a first phenotype (e.g., having a first disease) as compared to a biological sample from a subject or group of subjects having a second phenotype (e.g., not having the first disease). Such non-biomarker compounds may, however, be biomarkers in a biological sample from a subject or a group of subjects having a third phenotype (e.g., having a second disease) as compared to the first phenotype (e.g., having the first disease) or the second phenotype (e.g., not having the first disease).

“Metabolite”, or “small molecule”, means organic and inorganic molecules which are present in a cell. The term does not include large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weights of over 2,000, 3,000, 4,000, 5,000, 6,000, 7,000, 8,000, 9,000, or 10,000). The small molecules of the cell are generally found free in solution in the cytoplasm or in other organelles, such as the mitochondria, where they form a pool of intermediates which can be metabolized further or used to generate large molecules, called macromolecules. The term “small molecules” includes signaling molecules and intermediates in the chemical reactions that transform energy derived from food into usable forms. Examples of small molecules include sugars, fatty acids, amino acids, nucleotides, intermediates formed during cellular processes, and other small molecules found within the cell.

“Metabolic profile”, or “small molecule profile”, means a complete or partial inventory of small molecules within a targeted cell, tissue, organ, organism, or fraction thereof (e.g., cellular compartment). The inventory may include the quantity and/or type of small molecules present. The “small molecule profile” may be determined using a single technique or multiple different techniques.

“Metabolome” means all of the small molecules present in a given organism.

“Prostate cancer” refers to a disease in which cancer develops in the prostate, a gland in the male reproductive system. “Low grade” or “lower grade” prostate cancer refers to non-metastatic prostate cancer, including malignant tumors with low potential for metastisis (i.e. prostate cancer that is considered to be “less aggressive”). Cancer tumors that are confined to the prostate (i.e. organ-confined, OC) are considered to be less aggressive prostate cancer. “High grade” or “higher grade” prostate cancer refers to prostate cancer that has metastasized in a subject, including malignant tumors with high potential for metastasis (prostate cancer that is considered to be “aggressive”). Cancer tumors that are not confined to the prostate (i.e. non-organ-confined, NOC) are considered to be aggressive prostate cancer. Tumors that are confined to the prostate (i.e., organ confined tumors) are considered to be less aggressive than tumors which are not confined to the prostate (i.e., non-organ confined tumors). “Aggressive” prostate cancer progresses, recurs and/or is the cause of death. Aggressive cancer may be characterized by one or more of the following: non-organ confined (NOC), association with extra capsular extensions (ECE), association with seminal vesicle invasion (SVI), association with lymph node invasion (LN), association with a Gleason Score major or Gleason Score minor of 4, and/or association with a Gleason Score Sum of 8 or higher. In contrast “less aggressive” cancer is confined to the prostate (organ confined, OC) and is not associated with extra capsular extensions (ECE), seminal vesicle invasion (SVI), lymph node invasion (LN), a Gleason Score major or Gleason Score minor of 4, or a Gleason Score Sum of 8 or higher.

### I. Biomarkers

The prostate cancer biomarkers described herein were discovered using metabolomic profiling techniques. Such metabolomic profiling techniques are described in more detail in the Examples set forth below as well as in U.S. Pat. Nos. 7,005,255, 7,329,489; 7,550,258; 7,550,260; 7,553,616; 7,635,556; 7,682,783; 7,682,784; 7,910,301; 6,947,453; 7,433,787; 7,561,975; 7,884,318, the entire contents of which are hereby incorporated herein by reference.

Generally, metabolic profiles were determined for biological samples from human subjects diagnosed with prostate cancer, the human subjects were diagnosed with lower grade prostate cancer (e.g., organ-confined tumor) or were diagnosed with metastatic/high grade prostate cancer (e.g., non-organ confined tumor). The metabolic profile for biological samples from a subject having prostate cancer was compared to the metabolic profile for biological samples from the one or more other groups of subjects. Those molecules differentially present, including those molecules differentially present at a level that is statistically significant, in the metabolic profile of tumor samples from subjects with aggressive prostate cancer as compared to another group (e.g., subjects diagnosed with less aggressive prostate cancer) were identified as biomarkers to distinguish those groups. In addition, those molecules differentially present, including those molecules differentially present at a level that is statistically significant, in the metabolic profile of non-tumor samples (i.e., non-cancerous tissue adjacent to a cancer tumor) from subjects with low grade prostate cancer as compared to high grade prostate cancer were also identified as biomarkers to distinguish those groups.

The biomarkers are discussed in more detail herein. The biomarkers that were discovered correspond with the following group(s):
Biomarkers for distinguishing subjects having prostate cancer vs. control subjects not diagnosed with prostate cancer (see Tables 1A, 1B, 3A, 3B, and 8); and
Biomarkers for distinguishing subjects having aggressive prostate cancer from subjects with less aggressive prostate cancer (see Tables 1A, 1B, 3A, 3B, 5A, 5B, 7A, 7B, and 10);
   although the biomarkers in Tables 5A, 5B, 7A, 7B, and 10 may also be used to distinguish subjects having prostate cancer vs. control subjects not diagnosed with prostate cancer, and the biomarkers in Table 8 may also be used to distinguish subjects having aggressive prostate cancer from subjects with less aggressive prostate cancer.

### IIA. Diagnosis of Prostate Cancer

The identification of biomarkers for prostate cancer allows for the diagnosis of (or for aiding in the diagnosis of) prostate cancer in subjects presenting one or more symptoms of prostate cancer. A method of diagnosing (or aiding in diagnosing) whether a subject has prostate cancer comprises (1) analyzing a biological sample from a subject to determine the level(s) of one or more biomarkers of prostate cancer in the sample and (2) comparing the level(s) of the one or more biomarkers in the sample to prostate cancer-positive and/or prostate cancer-negative reference levels of the one or more biomarkers in order to diagnose (or aid in the diagnosis of) whether the subject has prostate cancer. The one or more biomarkers that are used are selected from Tables 1A, 1B, 3A, 3B, 5A, 5B, 7A, and/or 7B and combinations thereof. In one aspect, the one or more biomarkers may be selected from Tables 1A, 1B, 3A, 3B, and 8. When such a method is used to aid in the diagnosis of prostate cancer, the results of the method may be used along with other methods (or the results thereof) useful in the clinical determination of whether a subject has prostate cancer.

Any suitable method may be used to analyze the biological sample in order to determine the level(s) of the one or more biomarkers in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, and combinations thereof. Further, the level(s) of the one or more biomarkers may be measured indirectly, for example, by using an assay that measures the level of a compound (or compounds) that correlates with the level of the biomarker(s) that are desired to be measured.

The levels of one or more of the biomarkers of Tables 1A, 1B, 3A, 3B, 5A, 5B, 7A, 7B, 8 and/or 10 may be determined in the methods of diagnosing and methods of aiding in diagnosing whether a subject has prostate cancer. For example, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, etc., including a combination of all of the biomarkers in Tables 1A, 1B, 3A, 3B, 5A, 5B, 7A, 7, 8, and/or 10 and combinations thereof or any fraction thereof, may be determined and used in such methods. Determining levels of combinations of the biomarkers may allow greater sensitivity and specificity in diagnosing prostate cancer and aiding in the diagnosis of prostate cancer, and may allow better differentiation of prostate cancer from other prostate disorders (e.g. benign prostatic hypertrophy (BPH), prostatitis, etc.) or other cancers that may have similar or overlapping biomarkers to prostate cancer (as compared to a subject not having prostate cancer). For example, ratios of the levels of certain biomarkers (and non-biomarker compounds) in biological samples may allow greater sensitivity and specificity in diagnosing prostate cancer and aiding in the diagnosis of prostate cancer and may allow better differentiation of prostate cancer from other cancers or other disorders of the prostate that may have similar or overlapping biomarkers to prostate cancer (as compared to a subject not having prostate cancer).

One or more biomarkers that are specific for diagnosing prostate cancer (or aiding in diagnosing prostate cancer) in a certain type of sample (e.g., prostate tissue sample, urine sample, or blood plasma sample) may also be used. For example, when the biological sample is prostate tissue, one or more biomarkers listed in Tables 1A, 1B, 3A, 3B, 5A, 5B, 7A, 7B, and/or 10, may be used to diagnose (or aid in diagnosing) whether a subject has prostate cancer. As another example, when the biological sample is urine (or DRE urine), one or more biomarkers listed in Table 8 may be used to diagnose (or aid in diagnosing) whether a subject has prostate cancer.

After the level(s) of the one or more biomarkers in the sample are determined, the level(s) are compared to prostate cancer-positive and/or prostate cancer-negative reference levels to aid in diagnosing or to diagnose whether the subject has prostate cancer. Levels of the one or more biomarkers in a sample matching the prostate cancer-positive reference levels (e.g., levels that are the same as the reference levels, substantially the same as the reference levels, above and/or below the minimum and/or maximum of the reference levels, and/or within the range of the reference levels) are indicative of a diagnosis of prostate cancer in the subject. Levels of the one or more biomarkers in a sample matching the prostate cancer-negative reference levels (e.g., levels that are the same as the reference levels, substantially the same as the reference levels, above and/or below the minimum and/or maximum of the reference levels, and/or within the range of the reference levels) are indicative of a diagnosis of no prostate cancer in the subject. In addition, levels of the one or more biomarkers that are differentially present (especially at a level that is statistically significant) in the sample as compared to prostate cancer-negative reference levels are indicative of a diagnosis of prostate cancer in the subject. Levels of the one or more biomarkers that are differentially present (especially at a level that is statistically significant) in the sample as compared to prostate cancer-positive reference levels are indicative of a diagnosis of no prostate cancer in the subject.

The level(s) of the one or more biomarkers may be compared to prostate cancer-positive and/or prostate cancer-negative reference levels using various techniques, including a simple comparison (e.g., a manual comparison) of the level(s) of the one or more biomarkers in the biological sample to prostate cancer-positive and/or prostate cancer-negative reference levels. The level(s) of the one or more biomarkers in the biological sample may also be compared to prostate cancer-positive and/or prostate cancer-negative reference levels using one or more statistical analyses (e.g., t-test, Welch's T-test, Wilcoxon's rank sum test, random forest).

In addition, the biological samples may be analyzed to determine the level(s) of one or more non-biomarker compounds. The level(s) of such non-biomarker compounds may also allow differentiation of prostate cancer from other prostate disorders that may have similar or overlapping biomarkers to prostate cancer (as compared to a subject not having a prostate disorder). For example, a known non-biomarker compound present in biological samples of subjects having prostate cancer and subjects not having prostate cancer could be monitored to verify a diagnosis of prostate cancer as compared to a diagnosis of another prostate disorder when biological samples from subjects having the prostate disorder do not have the non-biomarker compound.

The methods of diagnosing (or aiding in diagnosing) whether a subject has prostate cancer may also be conducted specifically to diagnose (or aid in diagnosing) whether a subject has less aggressive prostate cancer and/or high aggressive prostate cancer. Such methods comprise (1) analyzing a biological sample from a subject to determine the level(s) of one or more biomarkers of less aggressive prostate cancer (and/or high aggressove prostate cancer) in the sample and (2) comparing the level(s) of the one or more biomarkers in the sample to less aggressive prostate cancer-positive and/or less aggressive prostate cancer-negative reference levels (or high aggressive prostate cancer-positive and/or high aggressive prostate cancer-negative reference levels) in order to diagnose (or aid in the diagnosis of) whether the subject has less aggressive prostate cancer (or high aggressive prostate cancer). Biomarker specific for low grade prostate cancer are listed in Tables 1, 3, 7 and biomarkers specific for high grade prostate cancer are listed in Tables 1, 3, 7.

IIB. Methods of Distinguishing Less Aggressive Prostate Cancer (Low Grade) from More Aggressive Prostate Cancer (High Grade)

The identification of biomarkers for distinguishing less aggressive prostate cancer versus more aggressive prostate cancer allows less aggressive prostate cancer and aggressive prostate cancer to be distinguished in patients. The subjects can then be treated appropriately, with those subjects having more aggressive prostate cancer undergoing more aggressive treatment than those subjects with less aggressive prostate cancer. A method of distinguishing less aggressive prostate cancer from more aggressive prostate cancer in a subject having prostate cancer comprises (1) analyzing a biological sample from a subject to determine the level(s) in the sample of one or more biomarkers of less aggressive prostate cancer that distinguish over high aggressive prostate cancer and/or one or more biomarkers of high aggressive prostate cancer that distinguish over less aggressive prostate cancer, and (2) comparing the level(s) of the one or more biomarkers in the sample to less aggressive prostate cancer-positive reference levels that distinguish over high aggressive prostate cancer and/or high aggressive prostate cancer-positive reference levels that distinguish over less aggressive prostate cancer of the one or more biomarkers in order to determine whether the subject has less aggressive or high aggressive prostate cancer. The one or more biomarkers that are used are selected from Tables 1A, 1B, 3A, 3B, 5A, 5B, 7A, 7B, 8, and/or 10 and combinations thereof.

In one aspect of the invention, the biomarkers that are used are selected from Tables 1A, 1B, 3A, 3B, 5A, 5B, 7A, 7B, and/or 10 and combinations thereof.

In another aspect of the invention the one or more biomarkers that are used are selected from putrescine, lactate, 5,6-dihydrouracil, 10-nonadecenoate, NAD+, spermine, N-acetylputrescine, succinylcarnitine, 3-(4-hydroxyphenyl)lactate, 2-palmitoylglycerophosphoethanolamine, spermidine, glycerol-2-phosphate, glycylvaline, and/or phosphoethanolamine.

In an aspect of the invention, the more aggressive cancer is associated with extracapsular extensions (ECE) and the biomarker metabolites are selected from putrescine, lactate, 5,6-dihydrouracil, 10-nonadecenoate, NAD+, spermine, and/or N-acetylputrescine.

In an aspect of the invention, the more aggressive cancer is associated with seminal vesicle invasion (SVI) and the biomarkers are selected from putrescine, glycerol-2-phosphate, and/or glycylvaline.

In an aspect of the invention, the more aggressive cancer is associated with lymph node invasion and the biomarkers are selected from phosphoethanolamine, putrescine, and/or spermidine.

In an aspect of the invention, the more aggressive cancer is associated with a Gleason Score (GS) greater than 8 and the biomarkers are selected from succinylcarnitine, 3-(4-hydroxyphenyl)lactate, 2-palmitoylglycerophosphoethanolamine, lactate, and/or spermidine.

Any suitable method may be used to analyze the biological sample in order to determine the level(s) of the one or more biomarkers in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, and combinations thereof. Further, the level(s) of the one or more biomarkers may be measured indirectly, for example, by using an assay that measures the level of a compound (or compounds) that correlates with the level of the biomarker(s) that are desired to be measured.

The levels of one or more of the biomarkers of Tables 1A, 1B, 3A, 3B, 5A, 5B, 7A, 7B, 8, and/or 10 may be determined in the methods of diagnosing and methods of aiding in diagnosing whether a subject has prostate cancer. For example, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, etc., including a combination of all of the biomarkers in Tables 1A, 1B, 3A, 3B, 5A, 5B, 7A, 7B, 8, and/or 10 or any fraction thereof, may be determined and used in such methods. Determining levels of combinations of the biomarkers may allow greater sensitivity and specificity in distinguishing between low aggressive and high aggressive prostate cancer.

One or more biomarkers that are specific for distinguishing between less aggressive and high aggressive prostate cancer in a certain type of sample (e.g., prostate tissue sample, urine sample, or blood plasma sample) may also be used. For example, when the biological sample is prostate tissue, one or more biomarkers listed in Tables 1A, 1B, 3A, 3B, 5A, 5B, 7A, 7B, and/or 10 may be used. As another example, when the biological sample is urine (or DRE urine), one or more biomarkers listed in Table 8 may be used.

After the level(s) of the one or more biomarkers in the sample are determined, the level(s) are compared to less aggressive prostate cancer-positive reference levels that distinguish over high aggressive prostate cancer-negative and/or high aggressive prostate cancer-positive reference levels that distinguish over less aggressive prostate cancer of the one or more biomarkers in order to determine whether the subject has less aggressive or high aggressive prostate cancer. Levels of the one or more biomarkers in a sample matching the less aggressive prostate cancer-positive reference levels that distinguish over high aggressive prostate cancer (e.g., levels that are the same as the reference levels, substantially the same as the reference levels, above and/or below the minimum and/or maximum of the reference levels, and/or within the range of the reference levels) are indicative of less aggressive prostate cancer in the subject. Levels of the one or more biomarkers in a sample matching the high aggressive prostate cancer-positive reference levels that distinguish over low aggressive prostate cancer (e.g., levels that are the same as the reference levels, substantially the same as the reference levels, above and/or below the minimum and/or maximum of the reference levels, and/or within the range of the reference levels) are indicative of high-aggressive prostate cancer in the subject. If the level(s) of the one or more biomarkers are more similar to the less aggressive prostate cancer-positive reference levels that distinguish over high aggressive prostate cancer (or less similar to the high aggressive prostate cancer-positive reference levels), then the results are indicative of less aggressive prostate cancer in the subject. If the level(s) of the one or more biomarkers are more similar to the high aggressive prostate cancer-positive reference levels that distinguish over less aggressive prostate cancer (or less similar to the less aggressive prostate cancer-positive reference levels), then the results are indicative of high aggressive prostate cancer in the subject.

The level(s) of the one or more biomarkers may be compared to less aggressive prostate cancer-positive reference levels that distinguish over high aggressive prostate cancer and/or high aggressive prostate cancer-positive reference levels that distinguish over less aggressive prostate cancer using various techniques, including a simple comparison (e.g., a manual comparison) of the level(s) of the one or more biomarkers in the biological sample to less aggressive prostate cancer-positive and/or high aggressive prostate cancer-positive reference levels. The level(s) of the one or more biomarkers in the biological sample may also be compared to less aggressive prostate cancer-positive reference levels that distinguish over high aggressive prostate cancer and/or high aggressive prostate cancer-positive reference levels that distinguish over less aggressive prostate cancer using one or more statistical analyses (e.g., t-test, Welch's T-test, Wilcoxon's rank sum test, random forest).

In addition, the biological samples may be analyzed to determine the level(s) of one or more non-biomarker compounds. The level(s) of such non-biomarker compounds may also allow differentiation of less aggressive prostate cancer from high aggressive prostate cancer.

### III. Methods of Determining Predisposition to Prostate Cancer

The identification of biomarkers for prostate cancer also allows for the determination of whether a subject having no symptoms of prostate cancer is predisposed to developing prostate cancer. A method of determining whether a subject having no symptoms of prostate cancer is predisposed to developing prostate cancer comprises (1) analyzing a biological sample from a subject to determine the level(s) of one or more biomarkers listed in Tables 1A, 1B, 3A, 3B, 5A, 5B, 7A, 7B, 8, and/or 10 in the sample and (2) comparing the level(s) of the one or more biomarkers in the sample to prostate cancer-positive and/or prostate cancer-negative reference levels of the one or more biomarkers in order to determine whether the subject is predisposed to developing prostate cancer. The results of the method may be used along with other methods (or the results thereof) useful in the clinical determination of whether a subject is predisposed to developing prostate cancer.

As described above in connection with methods of diagnosing (or aiding in the diagnosis of) prostate cancer, any suitable method may be used to analyze the biological sample in order to determine the level(s) of the one or more biomarkers in the sample.

As with the methods of diagnosing (or aiding in the diagnosis of) prostate cancer described above, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, etc., including a combination of all of the biomarkers in Tables 1A, 1B, 3A, 3B, 5A, 5B, 7A, 7B, 8, and/or 10 or any fraction thereof, may be determined and used in methods of determining whether a subject having no symptoms of prostate cancer is predisposed to developing prostate cancer.

After the level(s) of the one or more biomarkers in the sample are determined, the level(s) are compared to prostate cancer-positive and/or prostate cancer-negative reference levels in order to predict whether the subject is predisposed to developing prostate cancer. Levels of the one or more biomarkers in a sample matching the prostate cancer-positive reference levels (e.g., levels that are the same as the reference levels, substantially the same as the reference levels, above and/or below the minimum and/or maximum of the reference levels, and/or within the range of the reference levels) are indicative of the subject being predisposed to developing prostate cancer. Levels of the one or more biomarkers in a sample matching the prostate cancer-negative reference levels (e.g., levels that are the same as the reference levels, substantially the same as the reference levels, above and/or below the minimum and/or maximum of the reference levels, and/or within the range of the reference levels) are indicative of the subject not being predisposed to developing prostate cancer. In addition, levels of the one or more biomarkers that are differentially present (especially at a level that is statistically significant) in the sample as compared to prostate cancer-negative reference levels are indicative of the subject being predisposed to developing prostate cancer. Levels of the one or more biomarkers that are differentially present (especially at a level that is statistically significant) in the sample as compared to prostate cancer-positive reference levels are indicative of the subject not being predisposed to developing prostate cancer.

Furthermore, it may also be possible to determine reference levels specific to assessing whether or not a subject that does not have prostate cancer is predisposed to developing prostate cancer. For example, it may be possible to determine reference levels of the biomarkers for assessing different degrees of risk (e.g., low, medium, high) in a subject for developing prostate cancer. Such reference levels could be used for comparison to the levels of the one or more biomarkers in a biological sample from a subject.

As with the methods described above, the level(s) of the one or more biomarkers may be compared to prostate cancer-positive and/or prostate cancer-negative reference levels using various techniques, including a simple comparison, one or more statistical analyses, and combinations thereof.

As with the methods of diagnosing (or aiding in diagnosing) whether a subject has prostate cancer, the methods of determining whether a subject having no symptoms of prostate cancer is predisposed to developing prostate cancer may further comprise analyzing the biological sample to determine the level(s) of one or more non-biomarker compounds.

The methods of determining whether a subject having no symptoms of prostate cancer is predisposed to developing prostate cancer may also be conducted specifically to determine whether a subject having no symptoms of prostate cancer is predisposed to developing less aggressive prostate cancer and/or high aggressive prostate cancer. Biomarker specific for less aggressive prostate cancer are listed in Tables 1, 3, 5, 7, and 10 and biomarkers specific for high aggressive prostate cancer are listed in Tables 1, 3, 5, 7, and 10.

In addition, methods of determining whether a subject having less aggressive prostate cancer is predisposed to developing high aggressive prostate cancer may be conducted using one or more biomarkers selected from Tables 1A, 1B, 3A, 3B, 5A, 5B, 7A, 7B, 8, and/or 10.

### IV. Methods of Monitoring Progression/Regression of Prostate Cancer

The identification of biomarkers for prostate cancer also allows for monitoring progression/regression of prostate cancer in a subject. A method of monitoring the progression/regression of prostate cancer in a subject comprises (1) analyzing a first biological sample from a subject to determine the level(s) of one or more biomarkers for prostate cancer selected from Tables 1A, 1B, 3A, 3B, 5A, 5B, 7A, 7B, 8, and/or 10, the first sample obtained from the subject at a first time point, (2) analyzing a second biological sample from a subject to determine the level(s) of the one or more biomarkers, the second sample obtained from the subject at a second time point, and (3) comparing the level(s) of one or more biomarkers in the first sample to the level(s) of the one or more biomarkers in the second sample in order to monitor the progression/regression of prostate cancer in the subject. The results of the method are indicative of the course of prostate cancer (i.e., progression or regression, if any change) in the subject.

The change (if any) in the level(s) of the one or more biomarkers over time may be indicative of progression or regression of prostate cancer in the subject. In order to characterize the course of prostate cancer in the subject, the level(s) of the one or more biomarkers in the first sample, the level(s) of the one or more biomarkers in the second sample, and/or the results of the comparison of the levels of the biomarkers in the first and second samples may be compared to prostate cancer-positive, prostate cancer-negative, less aggressive prostate cancer-positive, less aggressive prostate cancer-negative, high-aggressive prostate cancer-positive, and/or high aggressive prostate cancer-negative reference levels as well as less aggressive prostate cancer-positive reference levels that distinguish over high aggressive prostate cancer and/or high aggressive prostate cancer-positive reference levels that distinguish over low aggressive prostate cancer. If the comparisons indicate that the level(s) of the one or more biomarkers are increasing or decreasing over time (e.g., in the second sample as compared to the first sample) to become more similar to the prostate cancer-positive reference levels (or less similar to the prostate cancer-negative reference levels), to the high aggressive prostate cancer reference levels, or, when the subject initially has less aggressive prostate cancer, to the high aggressive prostate cancer-positive reference levels that distinguish over less aggressive prostate cancer, then the results are indicative of prostate cancer progression. If the comparisons indicate that the level(s) of the one or more biomarkers are increasing or decreasing over time to become more similar to the prostate cancer-negative reference levels (or less similar to the prostate cancer-positive reference levels), or, when the subject initially has high aggressive prostate cancer, to less aggressive prostate cancer reference levels and/or to less aggressive prostate cancer-positive reference levels that distinguish over high aggressive prostate cancer, then the results are indicative of prostate cancer regression.

As with the other methods described herein, the comparisons made in the methods of monitoring progression/regression of prostate cancer in a subject may be carried out using various techniques, including simple comparisons, one or more statistical analyses, and combinations thereof.

The results of the method may be used along with other methods (or the results thereof) useful in the clinical monitoring of progression/regression of prostate cancer in a subject.

As described above in connection with methods of diagnosing (or aiding in the diagnosis of) prostate cancer, any suitable method may be used to analyze the biological samples in order to determine the level(s) of the one or more biomarkers in the samples. In addition, the level(s) one or more biomarkers, including a combination of all of the biomarkers in Tables 1A, 1B, 3A, 3B, 5A, 5B, 7A, 7B, 8, and/or 10 or any fraction thereof, may be determined and used in methods of monitoring progression/regression of prostate cancer in a subject.

Such methods could be conducted to monitor the course of prostate cancer in subjects having prostate cancer or could be used in subjects not having prostate cancer (e.g., subjects suspected of being predisposed to developing prostate cancer) in order to monitor levels of predisposition to prostate cancer.

### V. Methods of Assessing Efficacy of Compositions for Treating Prostate Cancer

The identification of biomarkers for prostate cancer also allows for assessment of the efficacy of a composition for treating prostate cancer as well as the assessment of the relative efficacy of two or more compositions for treating prostate cancer. Such assessments may be used, for example, in efficacy studies as well as in lead selection of compositions for treating prostate cancer.

A method of assessing the efficacy of a composition for treating prostate cancer comprises (1) analyzing, from a subject having prostate cancer and currently or previously being treated with a composition, a biological sample to determine the level(s) of one or more biomarkers selected from Tables 1A, 1B, 3A, 3B, 5A, 5B, 7A, 7B, 8, and/or 10, and (2) comparing the level(s) of the one or more biomarkers in the sample to (a) level(s) of the one or more biomarkers in a previously-taken biological sample from the subject, wherein the previously-taken biological sample was obtained from the subject before being treated with the composition, (b) prostate cancer-positive reference levels (including less aggressive prostate cancer-positive and/or high aggressive prostate cancer-positive reference levels) of the one or more biomarkers, (c) prostate cancer-negative reference levels (including less aggressive prostate cancer-negative and/or high aggressive prostate cancer-negative reference levels) of the one or more biomarkers, (d) less aggressive prostate cancer-positive reference levels that distinguish over high aggressive prostate cancer, and/or (e) high aggressive prostate cancer-positive reference levels that distinguish over less aggressive prostate cancer. The results of the comparison are indicative of the efficacy of the composition for treating prostate cancer.

Thus, in order to characterize the efficacy of the composition for treating prostate cancer, the level(s) of the one or more biomarkers in the biological sample are compared to (1) prostate cancer-positive reference levels, (2) prostate cancer-negative reference levels, (3) previous levels of the one or more biomarkers in the subject before treatment with the composition, (4) less aggressive prostate cancer-positive reference levels that distinguish over high aggressive prostate cancer, and/or (5) high aggressive prostate cancer-positive reference levels that distinguish over less aggressive prostate cancer.

When comparing the level(s) of the one or more biomarkers in the biological sample (from a subject having prostate cancer and currently or previously being treated with a composition) to prostate cancer-positive reference levels and/or prostate cancer-negative reference levels, level(s) in the sample matching the prostate cancer-negative reference levels (e.g., levels that are the same as the reference levels, substantially the same as the reference levels, above and/or below the minimum and/or maximum of the reference levels, and/or within the range of the reference levels) are indicative of the composition having efficacy for treating prostate cancer. Levels of the one or more biomarkers in the sample matching the prostate cancer-positive reference levels (e.g., levels that are the same as the reference levels, substantially the same as the reference levels, above and/or below the minimum and/or maximum of the reference levels, and/or within the range of the reference levels) are indicative of the composition not having efficacy for treating prostate cancer. The comparisons may also indicate degrees of efficacy for treating prostate cancer based on the level(s) of the one or more biomarkers.

When comparing the level(s) of the one or more biomarkers in the biological sample (from a subject having high aggressive prostate cancer and currently or previously being treated with a composition) less aggressive prostate cancer-positive reference levels that distinguish over high aggressive prostate cancer and/or high aggressive prostate cancer-positive reference levels that distinguish over less aggressive prostate cancer, level(s) in the sample matching the less aggressive prostate cancer-positive reference levels that distinguish over high aggressive prostate cancer (e.g., levels that are the same as the reference levels, substantially the same as the reference levels, above and/or below the minimum and/or maximum of the reference levels, and/or within the range of the reference levels) are indicative of the composition having efficacy for treating prostate cancer. Levels of the one or more biomarkers in the sample matching the high aggressive prostate cancer-positive reference levels that distinguish over less aggressive prostate cancer (e.g., levels that are the same as the reference levels, substantially the same as the reference levels, above and/or below the minimum and/or maximum of the reference levels, and/or within the range of the reference levels) are indicative of the composition not having efficacy for treating prostate cancer.

When the level(s) of the one or more biomarkers in the biological sample (from a subject having prostate cancer and currently or previously being treated with a composition) are compared to level(s) of the one or more biomarkers in a previously-taken biological sample from the subject before treatment with the composition, any changes in the level(s) of the one or more biomarkers are indicative of the efficacy of the composition for treating prostate cancer. That is, if the comparisons indicate that the level(s) of the one or more biomarkers have increased or decreased after treatment with the composition to become more similar to the prostate cancer-negative reference levels (or less similar to the prostate cancer-positive reference levels) or, when the subject initially has high aggressive prostate cancer, the level(s) have increased or decreased to become more similar to less aggressive prostate cancer-positive reference levels that distinguish over high aggressive prostate cancer (or less similar to the high aggressive prostate cancer-positive reference levels that distinguish over low aggressive prostate cancer), then the results are indicative of the composition having efficacy for treating prostate cancer. If the comparisons indicate that the level(s) of the one or more biomarkers have not increased or decreased after treatment with the composition to become more similar to the prostate cancer-negative reference levels (or less similar to the prostate cancer-positive reference levels) or, when the subject initially has high aggressive prostate cancer, the level(s) have not increased or decreased to become more similar to less aggressive prostate cancer-positive reference levels that distinguish over high aggressive prostate cancer (or less similar to the high aggressive prostate cancer-positive reference levels that distinguish over less aggressive prostate cancer), then the results are indicative of the composition not having efficacy for treating prostate cancer. The comparisons may also indicate degrees of efficacy for treating prostate cancer based on the amount of changes observed in the level(s) of the one or more biomarkers after treatment. In order to help characterize such a comparison, the changes in the level(s) of the one or more biomarkers, the level(s) of the one or more biomarkers before treatment, and/or the level(s) of the one or more biomarkers in the subject currently or previously being treated with the composition may be compared to prostate cancer-positive reference levels (including less aggressive and high aggressive prostate cancer-positive reference levels), prostate cancer-negative reference levels (including less aggressive and high aggressive prostate cancer-negative reference levels), less aggressive prostate cancer-positive reference levels that distinguish over high aggressive prostate cancer, and/or high aggressive prostate cancer-positive reference levels that distinguish over less aggressive prostate cancer.

Another method for assessing the efficacy of a composition in treating prostate cancer comprises (1) analyzing a first biological sample from a subject to determine the level(s) of one or more biomarkers selected from Tables 1A, 1B, 3A, 3B, 5A, 5B, 7A, 7B, 8, and/or 10, the first sample obtained from the subject at a first time point, (2) administering the composition to the subject, (3) analyzing a second biological sample from a subject to determine the level(s) of the one or more biomarkers, the second sample obtained from the subject at a second time point after administration of the composition, and (4) comparing the level(s) of one or more biomarkers in the first sample to the level(s) of the one or more biomarkers in the second sample in order to assess the efficacy of the composition for treating prostate cancer. As indicated above, if the comparison of the samples indicates that the level(s) of the one or more biomarkers have increased or decreased after administration of the composition to become more similar to the prostate cancer-negative reference levels (or less similar to the prostate cancer-positive reference levels) or, when the subject initially has high aggressive prostate cancer, if the level(s) have increased or decreased to become more similar to less aggressive prostate cancer-positive reference levels that distinguish over high aggressive prostate cancer (or less similar to the high aggressive prostate cancer-positive reference levels that distinguish over less aggressive prostate cancer), then the results are indicative of the composition having efficacy for treating prostate cancer. If the comparisons indicate that the level(s) of the one or more biomarkers have not increased or decreased after treatment with the composition to become more similar to the prostate cancer-negative reference levels (or less similar to the prostate cancer-positive reference levels) or, when the subject initially has high aggressive prostate cancer, the level(s) have not increased or decreased to become more similar to less aggressive prostate cancer-positive reference levels that distinguish over high aggressive prostate cancer (or less similar to the high aggressive prostate cancer-positive reference levels that distinguish over less aggressive prostate cancer), then the results are indicative of the composition not having efficacy for treating prostate cancer. The comparison may also indicate a degree of efficacy for treating prostate cancer based on the amount of changes observed in the level(s) of the one or more biomarkers after administration of the composition as discussed above.

A method of assessing the relative efficacy of two or more compositions for treating prostate cancer comprises (1) analyzing, from a first subject having prostate cancer and currently or previously being treated with a first composition, a first biological sample to determine the level(s) of one or more biomarkers selected from Tables 1A, 1B, 3A, 3B, 5A, 5B, 7A, 7B, 8 and/or 10 (2) analyzing, from a second subject having prostate cancer and currently or previously being treated with a second composition, a second biological sample to determine the level(s) of the one or more biomarkers, and (3) comparing the level(s) of one or more biomarkers in the first sample to the level(s) of the one or more biomarkers in the second sample in order to assess the relative efficacy of the first and second compositions for treating prostate cancer. The results are indicative of the relative efficacy of the two compositions, and the results (or the levels of the one or more biomarkers in the first sample and/or the level(s) of the one or more biomarkers in the second sample) may be compared to prostate cancer-positive reference levels (including less aggressive and high aggressive prostate cancer-positive reference levels), prostate cancer-negative reference levels (including less aggressive and high aggressive prostate cancer-negative reference levels), less aggressive prostate cancer-positive reference levels that distinguish over high aggressive prostate cancer, and/or high aggressive prostate cancer-positive reference levels that distinguish over less aggressive prostate cancer to aid in characterizing the relative efficacy.

Each of the methods of assessing efficacy may be conducted on one or more subjects or one or more groups of subjects (e.g., a first group being treated with a first composition and a second group being treated with a second composition).

As with the other methods described herein, the comparisons made in the methods of assessing efficacy (or relative efficacy) of compositions for treating prostate cancer may be carried out using various techniques, including simple comparisons, one or more statistical analyses, and combinations thereof. Any suitable method may be used to analyze the biological samples in order to determine the level(s) of the one or more biomarkers in the samples. In addition, the level(s) of one or more biomarkers, including a combination of all of the biomarkers in Tables 1A, 1B, 3A, 3B, 5A, 5B, 7A, 7B, 8, and/or 10 or any fraction thereof, may be determined and used in methods of assessing efficacy (or relative efficacy) of compositions for treating prostate cancer.

Finally, the methods of assessing efficacy (or relative efficacy) of one or more compositions for treating prostate cancer may further comprise analyzing the biological sample to determine the level(s) of one or more non-biomarker compounds. The non-biomarker compounds may then be compared to reference levels of non-biomarker compounds for subjects having (or not having) prostate cancer.

VI. Methods of Screening a Composition for Activity in Modulating Biomarkers Associated with Prostate Cancer

The identification of biomarkers for prostate cancer also allows for the screening of compositions for activity in modulating biomarkers associated with prostate cancer, which may be useful in treating prostate cancer. Methods of screening compositions useful for treatment of prostate cancer comprise assaying test compositions for activity in modulating the levels of one or more biomarkers in Tables 1A, 1B, 3A, 3B, 5A, 5B, 7A, 7B, 8, and/or 10. Such screening assays may be conducted in vitro and/or in vivo, and may be in any form known in the art useful for assaying modulation of such biomarkers in the presence of a test composition such as, for example, cell culture assays, organ culture assays, and in vivo assays (e.g., assays involving animal models).

In one embodiment, a method for screening a composition for activity in modulating one or more biomarkers of prostate cancer comprises (1) contacting one or more cells with a composition, (2) analyzing at least a portion of the one or more cells or a biological sample associated with the cells to determine the level(s) of one or more biomarkers of prostate cancer selected from Tables 1A, 1B, 3A, 3B, 5A, 5B, 7A, 7B, 8, and/or 10; and (3) comparing the level(s) of the one or more biomarkers with predetermined standard levels for the one or more biomarkers to determine whether the composition modulated the level(s) of the one or more biomarkers. As discussed above, the cells may be contacted with the composition in vitro and/or in vivo. The predetermined standard levels for the one or more biomarkers may be the levels of the one or more biomarkers in the one or more cells in the absence of the composition. The predetermined standard levels for the one or more biomarkers may also be the level(s) of the one or more biomarkers in control cells not contacted with the composition.

In addition, the methods may further comprise analyzing at least a portion of the one or more cells or a biological sample associated with the cells to determine the level(s) of one or more non-biomarker compounds of prostate cancer. The levels of the non-biomarker compounds may then be compared to predetermined standard levels of the one or more non-biomarker compounds.

Any suitable method may be used to analyze at least a portion of the one or more cells or a biological sample associated with the cells in order to determine the level(s) of the one or more biomarkers (or levels of non-biomarker compounds). Suitable methods include chromatography (e.g., HPLC, gas chromatograph, liquid chromatography), mass spectrometry (e.g., MS, MS-MS), ELISA, antibody linkage, other immunochemical techniques, and combinations thereof. Further, the level(s) of the one or more biomarkers (or levels of non-biomarker compounds) may be measured indirectly, for example, by using an assay that measures the level of a compound (or compounds) that correlates with the level of the biomarker(s) (or non-biomarker compounds) that are desired to be measured.

### VII. Method of Identifying Potential Drug Targets

The identification of biomarkers for prostate cancer also allows for the identification of potential drug targets for prostate cancer. A method for identifying a potential drug target for prostate cancer comprises (1) identifying one or more biochemical pathways associated with one or more biomarkers for prostate cancer selected from Tables 1A, 1B, 3A, 3B, 5A, 5B, 7A, 7B, 8, and/or 10 and (2) identifying a protein (e.g., an enzyme) affecting at least one of the one or more identified biochemical pathways, the protein being a potential drug target for prostate cancer.

Another method for identifying a potential drug target for prostate cancer comprises (1) identifying one or more biochemical pathways associated with one or more biomarkers for prostate cancer selected from Tables 1A, 1B, 3A, 3B, 5A, 5B, 7A, 7B, 8, and/or 10 and one or more non-biomarker compounds of prostate cancer and (2) identifying a protein affecting at least one of the one or more identified biochemical pathways, the protein being a potential drug target for prostate cancer.

One or more biochemical pathways (e.g., biosynthetic and/or metabolic (catabolic) pathway) are identified that are associated with one or more biomarkers (or non-biomarker compounds). After the biochemical pathways are identified, one or more proteins affecting at least one of the pathways are identified. Preferably, those proteins affecting more than one of the pathways are identified.

A build-up of one metabolite (e.g., a pathway intermediate) may indicate the presence of a ‘block’ downstream of the metabolite and the block may result in a low/absent level of a downstream metabolite (e.g. product of a biosynthetic pathway). In a similar manner, the absence of a metabolite could indicate the presence of a ‘block’ in the pathway upstream of the metabolite resulting from inactive or non-functional enzyme(s) or from unavailability of biochemical intermediates that are required substrates to produce the product. Alternatively, an increase in the level of a metabolite could indicate a genetic mutation that produces an aberrant protein which results in the over-production and/or accumulation of a metabolite which then leads to an alteration of other related biochemical pathways and result in dysregulation of the normal flux through the pathway; further, the build-up of the biochemical intermediate metabolite may be toxic or may compromise the production of a necessary intermediate for a related pathway. It is possible that the relationship between pathways is currently unknown and this data could reveal such a relationship.

For example, the data indicates that metabolites in the biochemical pathways involving nitrogen excretion, amino acid metabolism, energy metabolism, oxidative stress, purine metabolism and bile acid metabolism are enriched in prostate cancer subjects. Further, polyamine levels are higher in cancer subjects, which indicates that the level and/or activity of the enzyme ornithine decarboxylase is increased. It is known that polyamines can act as mitotic agents and have been associated with free radical damage. These observations indicate that the pathways leading to the production of polyamines (or to any of the aberrant biomarkers) would provide a number of potential targets useful for drug discovery.

The proteins identified as potential drug targets may then be used to identify compositions that may be potential candidates for treating prostate cancer, including compositions for gene therapy.

### VIII. Methods of Treating Prostate Cancer

The identification of biomarkers for prostate cancer also allows for the treatment of prostate cancer. For example, in order to treat a subject having prostate cancer, an effective amount of one or more prostate cancer biomarkers that are lowered in prostate cancer as compared to a healthy subject not having prostate cancer may be administered to the subject. The biomarkers that may be administered may comprise one or more of the biomarkers in Tables 1A, 1B, 3A, 3B, 5A, 5B, 7A, 7B, 8, and/or 10 that are decreased in prostate cancer. In some embodiments, the biomarkers that are administered are one or more biomarkers listed in Tables 1A, 1B, 3A, 3B, 5A, 5B, 7A, 7B, 8, and/or 10 that are decreased in prostate cancer and that have a p-value less than 0.10. In other embodiments, the biomarkers that are administered are one or biomarkers listed in Tables 1A, 1B, 3A, 3B, 5A, 5B, 7A, 7B, 8, and/or 10 that are decreased in prostate cancer by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent).

### IX. Methods of Using the Prostate Cancer Biomarkers for Other Types of Cancer

It is believed that some of the biomarkers for major prostate cancer described herein may also be biomarkers for other types of cancer, including, for example, lung cancer or kidney cancer. Therefore, it is believed that at least some of the prostate cancer biomarkers may be used in the methods described herein for other types of cancer. That is, the methods described herein with respect to prostate cancer may also be used for diagnosing (or aiding in the diagnosis of) any type of cancer, methods of monitoring progression/regression of any type of cancer, methods of assessing efficacy of compositions for treating any type of cancer, methods of screening a composition for activity in modulating biomarkers associated with any type of cancer, methods of identifying potential drug targets for any type of cancer, and methods of treating any type of cancer. Such methods could be conducted as described herein with respect to prostate cancer.

### X. Methods of Using the Prostate Cancer Biomarkers for Other Prostate Disorders

It is believed that some of the biomarkers for prostate cancer described herein may also be biomarkers for prostate disorders (e.g. prostatitis, benign prostate hypertrophy (BHP)) in general. Therefore, it is believed that at least some of the prostate cancer biomarkers may be used in the methods described herein for prostate disorders in general. That is, the methods described herein with respect to prostate cancer may also be used for diagnosing (or aiding in the diagnosis of) a prostate disorder, methods of monitoring progression/regression of a prostate disorder, methods of assessing efficacy of compositions for treating a prostate disorder, methods of screening a composition for activity in modulating biomarkers associated with a prostate disorder, methods of identifying potential drug targets for prostate disorder, and methods of treating a prostate disorder. Such methods could be conducted as described herein with respect to prostate cancer.

### XI. Other Methods

Other methods of using the biomarkers discussed herein are also contemplated. For example, the methods described in U.S. Pat. No. 7,005,255, U.S. Pat. No. 7,329,489, U.S. Pat. No. 7,553,616, U.S. Pat. No. 7,550,260, U.S. Pat. No. 7,550,258, U.S. Pat. No. 7,635,556, U.S. patent application Ser. No. 11/728,826, U.S. patent application Ser. No. 12/463,690 and U.S. patent application Ser. No. 12/182,828 may be conducted using a small molecule profile comprising one or more of the biomarkers disclosed herein.

In any of the methods listed herein, the biomarkers that are used may be selected from those biomarkers in Tables 1A, 1B, 3A, or 3B, 5A, 5B, 7A, 7B, 8, and/or 10 having p-values of less than 0.05 and/or those biomarkers in Tables 1A, 1B, 3A, 3B, 5A, 5B, 7A, 7B, 8, and/or 10 having q-values of less than 0.10. The biomarkers that are used in any of the methods described herein may also be selected from those biomarkers in Tables 1A, 1B, 3A, 3B, 5A, 5B, 7A, 7B, 8, and/or 10 that are decreased in prostate cancer (as compared to the control) or that are decreased in remission (as compared to control or prostate cancer) by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent); and/or those biomarkers in Tables 1A, 1B, 3A, 3B, 5A, 5B, 7A, 7B, 8, and/or 10 that are increased in prostate cancer (as compared to the control or remission) or that are increased in remission (as compared to the control or prostate cancer) by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more.

### EXAMPLES

The invention will be further explained by the following illustrative examples that are intended to be non-limiting.

### I. General Methods

A. Identification of Metabolic Profiles for Prostate Cancer

Each sample was analyzed to determine the concentration of several hundred metabolites. Analytical techniques such as GC-MS (gas chromatography-mass spectrometry) and LC-MS (liquid chromatography-mass spectrometry) were used to analyze the metabolites. Multiple aliquots were simultaneously, and in parallel, analyzed, and, after appropriate quality control (QC), the information derived from each analysis was recombined. Every sample was characterized according to several thousand characteristics, which ultimately amount to several hundred chemical species. The techniques used were able to identify novel and chemically unnamed compounds.

B. Statistical Analysis

The data was analyzed using T-tests to identify molecules (either known, named metabolites or unnamed metabolites) present at differential levels in a definable population or subpopulation (e.g., biomarkers for prostate cancer biological samples compared to control biological samples or compared to patients in remission from prostate cancer) useful for distinguishing between the definable populations (e.g., prostate cancer and control, low aggressive prostate cancer and high aggressive prostate cancer). Other molecules (either known, named metabolites or unnamed metabolites) in the definable population or subpopulation were also identified.

Data was also analyzed using Random Forest Analysis. Random forests give an estimate of how well individuals in a new data set can be classified into existing groups. Random forest analysis creates a set of classification trees based on continual sampling of the experimental units and compounds. Then each observation is classified based on the majority votes from all the classification trees. In statistics, a classification tree classifies the observations into groups based on combinations of the variables (in this instance variables are metabolites or compounds). There are many variations on the algorithms used to create trees. A tree algorithm searches for the metabolite (compound) that provides the largest split between the two groups. This produces nodes. Then at each node, the metabolite that provides the best split is used and so on. If the node cannot be improved on, then it stops at that node and any observation in that node is classified as the majority group.

Random forests classify based on a large number (e.g. thousands) of trees. A subset of compounds and a subset of observations are used to create each tree. The observations used to create the tree are called the in-bag samples, and the remaining samples are called the out-of-bag samples. The classification tree is created from the in-bag samples, and the out-of-bag samples are predicted from this tree. To get the final classification for an observation, the “votes” for each group are counted based on the times it was an out-of-bag sample. For example, suppose observation 1 was classified as a “Control” by 2,000 trees, but classified as “Disease” by 3,000 trees. Using “majority wins” as the criterion, this sample is classified as “Disease.”

The results of the random forest are summarized in a confusion matrix. The rows correspond to the true grouping, and the columns correspond to the classification from the random forest. Thus, the diagonal elements indicate the correct classifications. A 50% error would occur by random chance for 2 groups, 66.67% error for three groups by random chance, etc. The “Out-of-Bag” (OOB) Error rate gives an estimate of how accurately new observations can be predicted using the random forest model (e.g., whether a sample is from a diseased subject or a control subject).

It is also of interest to see which variables are more “important” in the final classifications. The “importance plot” shows the top compounds ranked in terms of their importance. There are different criteria for ranking the importance, but the general idea is that removing an important variable will cause a greater decrease in accuracy than a variable that is less important. The most important identified biomarkers are presented in Tables 3A, 3B, 5A, 5B, 7A, and 7B.

C. Biomarker Identification

Various peaks identified in the analyses (e.g. GC-MS, LC-MS, MS-MS), including those identified as statistically significant, were subjected to a mass spectrometry based chemical identification process.

### Example 1

Biomarkers were discovered by (1) analyzing tissue samples from different groups of human subjects to determine the levels of metabolites in the samples and then (2) statistically analyzing the results to determine those metabolites that were differentially present in the two groups.

The tissue samples used for the analysis were 61 control tissues that were cancer free tissues derived from sections of prostate tissue not containing cancer cells (i.e. from cancerous prostate glands and that were determined to be free of cancerous cells), 46 prostate tissue samples from organ confined (T_OC) prostate cancer tumors (i.e. lower aggressive prostate cancer) and 25 prostate tissue samples from non-organ confined (T_NOC) prostate cancer tumors (i.e. high aggressive prostate cancer). After the levels of metabolites were determined, the data was analyzed using univariate T-tests (i.e., Welch's T-test).

T-tests were used to determine differences in the mean levels of metabolites between two populations (i.e., Prostate Cancer (T) vs. Control (C), High Aggressive (T_NOC) Prostate Cancer vs. Less Aggressive (T_OC) Prostate Cancer) and Adjacent tissue to High Aggressive Prostate Cancer (N_NOC) vs. Adjacent tissue to Less Aggressive Prostate Cancer Control (N_OC)).

### Biomarkers:

As listed below in Tables 1A and 1B, biomarkers were discovered that were differentially present between tissue samples from 1.) prostate cancer tumors and Control prostate tissue that was determined to be free of cancerous cells (i.e. sections of prostate tissue not containing cancerous cells from cancerous prostate glands removed from the patient), 2.) aggressive prostate tumors (i.e. tumors that were non-organ confined, NOC) and less aggressive prostate tumors (i.e. tumors that were organ confined, OC) and 3.) between NOC and OC cancer using non-cancer tissue adjacent to the NOC cancer tumor or the OC cancer tumor. The study was comprised of tissue collected from 25 subjects with non-organ-confined (NOC) prostate tumors and 46 subjects with (OC) organ-confined prostate cancer tumors.

Tables 1A and 1B include, for each listed biomarker, the p-value and the q-value determined in the statistical analysis of the data concerning the biomarkers and the ratio of the mean level of cancer samples as compared to the control mean level (Tables 1A and 1B, columns 3-5), the p-value and the q-value determined in the statistical analysis of the data concerning the biomarkers and the ratio of the mean level of the non-cancer tissue adjacent to high aggressive prostate cancer (N_NOC) mean level as compared to the non-cancer tissue adjacent to less aggressive (N_OC) mean level (Tables 1A and 1B, columns 6-8), and the p-value and the q-value determined in the statistical analysis of the data concerning the biomarkers and the ratio of the mean level of the cancer tumor from high aggressive prostate cancer (T_NOC) mean level as compared to the cancer tumor from lower aggressive prostate cancer (T_OC) mean level (Tables 1A and 1B, columns 9-11). The term “Isobar” as used in the tables indicates the compounds that could not be distinguished from each other on the analytical platform used in the analysis (i.e., the compounds in an isobar elute at nearly the same time and have similar (and sometimes exactly the same) quant ions, and thus cannot be distinguished).

Tables 1A and 1B. Prostate Cancer Biomarkers.

Legend: C, Control non-cancer tissue adjacent to cancer tissue; T, Tumor cancer tissue; N_NOC, Non-cancerous tissue adjacent to cancer tumor that is Non-Organ Confined; N_OC, Non-Cancerous tissue adjacent to cancer tumor that is Organ Confined; T_NOC, Tumor tissue that is Non-Organ Confined; T_OC, Tumor tissue that is Organ Confined

TABLE 1A

| | | | | Ratio | | N_NOC | Ratio | T_NOC | | |
|---|---|---|---|---|---|---|---|---|---|---|
| | | | | Cancer | N_NOC | VS | NOC/ | VS | T_NOC | Ratio |
| | | | | Tumor/ | VS | N_OC | OC | T_OC | VS | T_NOC/ |
| Comp | | C VS T P- | C VS T | Control | N_OC | Q- | Adja- | P- | T_OC Q- | T_OC |
| ID | Name | VALUE | Q-VALUE | (T/C) | P-VALUE | VALUE | cent | VALUE | VALUE | Tumor |
| 35439 | glutaroyl carnitine | 2.2E−13 | 1.076E−11 | 2.5428 | 0.7797 | 0.5723 | 0.9867 | 0.8926 | 0.3790 | 0.9852 |
| 1356 | nonadecanoate (19:0) | 2.9E−13 | 1.076E−11 | 1.8780 | 0.2707 | 0.3190 | 1.1401 | 0.0357 | 0.0412 | 1.3227 |
| 33972 | 10-nonadecenoate (19:1n9) | 6E−13 | 1.365E−11 | 1.9738 | 0.0022 | 0.0281 | 1.3829 | 0.0040 | 0.0129 | 1.4216 |
| 19324 | 1-stearoylglycerophosphoinositol | 1.5E−11 | 1.705E−10 | 1.7487 | 0.0362 | 0.1082 | 1.4025 | 0.0040 | 0.0129 | 1.4886 |
| 27728 | glycerol 2-phosphate | 2.1E−11 | 1.987E−10 | 2.0245 | 0.9731 | 0.6302 | 0.9925 | 0.0872 | 0.0760 | 1.2966 |
| 37459 | ergothioneine | 4.5E−11 | 3.407E−10 | 1.7200 | 0.2226 | 0.2852 | 1.1414 | 0.1806 | 0.1228 | 1.2472 |
| 36747 | deoxycarnitine | 6.7E−11 | 4.46E−10 | 1.3905 | 0.0464 | 0.1223 | 1.1204 | 0.0963 | 0.0801 | 1.1071 |
| 37097 | tryptophan betaine | 7.7E−11 | 4.879E−10 | 1.3584 | 0.1732 | 0.2401 | 1.0891 | 0.0997 | 0.0823 | 1.3327 |
| 37455 | glycerophosphoethanolamine | 3.7E−10 | 1.607E−09 | 2.1207 | 0.4521 | 0.4293 | 0.9035 | 0.2214 | 0.1401 | 1.0885 |
| 32452 | propionylcarnitine | 1.4E−09 | 4.967E−09 | 1.4653 | 0.0446 | 0.1201 | 1.2477 | 0.1466 | 0.1064 | 1.2842 |
| 18467 | eicosapentaenoate (EPA; 20:5n3) | 1.7E−09 | 5.666E−09 | 1.6414 | 0.1555 | 0.2282 | 1.2593 | 0.3180 | 0.1803 | 1.0792 |
| 32654 | 3-dehydrocarnitine | 1.9E−09 | 6.024E−09 | 1.2935 | 0.2482 | 0.3025 | 1.0944 | 0.1089 | 0.0883 | 1.1905 |
| 32412 | butyrylcarnitine | 3.2E−09 | 8.956E−09 | 1.4534 | 0.0771 | 0.1586 | 1.1538 | 0.0172 | 0.0280 | 1.2936 |
| 33587 | eicosenoate (20:1n9 or 11) | 3.4E−09 | 9.39E−09 | 1.7489 | 0.0105 | 0.0602 | 1.3890 | 0.0001 | 0.0019 | 1.6222 |
| 1638 | arginine | 3.8E−09 | 9.953E−09 | 1.6913 | 0.2783 | 0.3269 | 1.2801 | 0.0337 | 0.0399 | 1.4488 |
| 17805 | dihomo-linoleate (20:2n6) | 6.8E−09 | 1.646E−08 | 1.7543 | 0.0053 | 0.0447 | 1.4025 | 0.0006 | 0.0044 | 1.5861 |
| 15772 | ribitol | 7.7E−09 | 1.83E−08 | 1.6384 | 0.0002 | 0.0149 | 1.5060 | 0.0000 | 0.0002 | 1.7684 |
| 15720 | N-acetylglutamate | 8.5E−09 | 1.962E−08 | 0.6162 | 0.1223 | 0.2005 | 1.1085 | 0.8721 | 0.3726 | 0.9202 |
| 35305 | 1-palmitoylglycerophosphoinositol | 1.3E−08 | 2.796E−08 | 1.6574 | 0.1761 | 0.2420 | 1.2487 | 0.0095 | 0.0195 | 1.3145 |
| 19260 | 1-oleoylglycerophosphoserine | 2E−08 | 4.021E−08 | 1.4208 | 0.1119 | 0.1909 | 1.1938 | 0.0754 | 0.0698 | 1.2107 |
| 36593 | 2-linoleoylglycerophospho- | 2.1E−08 | 4.021E−08 | 1.6367 | 0.0140 | 0.0666 | 1.5003 | 0.0017 | 0.0077 | 1.5788 |
| | ethanolamine | | | | | | | | | |
| 1577 | 2-aminobutyrate | 2.6E−08 | 5.039E−08 | 1.2443 | 0.0346 | 0.1064 | 1.2226 | 0.0049 | 0.0141 | 1.3036 |
| 35433 | hydroxyisovaleroyl carnitine | 2.7E−08 | 5.146E−08 | 1.8954 | 0.1565 | 0.2285 | 1.2063 | 0.0456 | 0.0476 | 1.3227 |
| 33080 | N-ethylglycinexylidide | 3.3E−08 | 6.118E−08 | 1.4492 | 0.5567 | 0.4712 | 1.1998 | 0.0435 | 0.0459 | 1.5639 |
| 37948 | 2-oleoylglycerophosphoserine | 5.2E−08 | 9.064E−08 | 1.4802 | 0.0204 | 0.0832 | 1.3687 | 0.0134 | 0.0243 | 1.5106 |
| 32198 | acetylcarnitine | 9.6E−08 | 1.49E−07 | 1.2642 | 0.9134 | 0.6222 | 0.9900 | 0.9119 | 0.3806 | 0.9668 |
| 32635 | 1-linoleoylglycerophospho | 1.2E−07 | 1.762E−07 | 1.7959 | 0.3015 | 0.3392 | 1.2398 | 0.0321 | 0.0391 | 1.4287 |
| | ethanolamine | | | | | | | | | |
| 32415 | docosadienoate (22:2n6) | 2.3E−07 | 3.131E−07 | 1.5734 | 0.0006 | 0.0204 | 1.4680 | 0.0001 | 0.0020 | 1.6508 |
| 3141 | betaine | 2.8E−07 | 3.737E−07 | 1.2893 | 0.9353 | 0.6302 | 0.9951 | 0.4788 | 0.2438 | 0.9541 |
| 34437 | 1-stearoylglycerophosphoglycerol | 2.8E−07 | 3.737E−07 | 2.0348 | 0.0828 | 0.1640 | 1.5698 | 0.1718 | 0.1184 | 1.4281 |
| 35162 | UDP-N-acetylglucosamine | 3.5E−07 | 4.525E−07 | 1.9109 | 0.4994 | 0.4470 | 0.9214 | 0.9489 | 0.3900 | 1.0176 |
| 32504 | docosapentaenoate | 3.9E−07 | 5.061E−07 | 1.4968 | 0.0125 | 0.0646 | 1.4746 | 0.0082 | 0.0175 | 1.4506 |
| | (n3 DPA; 22:5n3) | | | | | | | | | |
| 34565 | 1-palmitoleoylglycerophospho- | 4.2E−07 | 5.327E−07 | 2.2261 | 0.6581 | 0.5234 | 1.0482 | 0.2032 | 0.1341 | 1.1898 |
| | ethanolamine | | | | | | | | | |
| 32417 | docosatrienoate (22:3n3) | 7.9E−07 | 9.345E−07 | 1.7688 | 0.0012 | 0.0251 | 1.5248 | 0.0011 | 0.0060 | 2.1304 |
| 33971 | 10-heptadecenoate (17:1n7) | 8.3E−07 | 9.742E−07 | 1.2217 | 0.0147 | 0.0667 | 1.1435 | 0.0826 | 0.0745 | 1.1477 |
| 37419 | 1-heptadecanoylglycerophospho- | 9.8E−07 | 1.118E−06 | 1.8968 | 0.1528 | 0.2263 | 1.2312 | 0.0327 | 0.0391 | 1.3785 |
| | ethanolamine | | | | | | | | | |
| 21127 | 1-palmitoylglycerol | 3.1E−06 | 3.298E−06 | 1.5124 | 0.9054 | 0.6194 | 1.0302 | 0.0246 | 0.0329 | 1.3123 |
| | (1-monopalmitin) | | | | | | | | | |
| 19323 | docosahexaenoate (DHA; 22:6n3) | 3.3E−06 | 3.369E−06 | 1.6100 | 0.0753 | 0.1578 | 1.3489 | 0.0434 | 0.0459 | 1.5001 |
| 15506 | choline | 3.7E−06 | 3.725E−06 | 1.1487 | 0.0560 | 0.1310 | 1.0781 | 0.0027 | 0.0105 | 1.1544 |
| 35718 | dihomo-linolenate (20:3n3 or n6) | 4.2E−06 | 4.158E−06 | 1.6088 | 0.0443 | 0.1201 | 1.3770 | 0.0067 | 0.0162 | 1.5242 |
| 2134 | flavin adenine dinucleotide (FAD) | 4.8E−06 | 4.665E−06 | 1.2276 | 0.6157 | 0.5021 | 1.0230 | 0.4335 | 0.2269 | 1.0488 |
| 34035 | linolenate [alpha or gamma; | 9.8E−06 | 8.992E−06 | 1.3425 | 0.0305 | 0.0983 | 1.3680 | 0.0180 | 0.0286 | 1.4184 |
| | (18:3n3 or 6)] | | | | | | | | | |
| 33487 | glutamate, gamma-methyl ester | 1.1E−05 | 9.867E−06 | 1.7460 | 0.1457 | 0.2198 | 0.7586 | 0.2105 | 0.1368 | 0.7030 |
| 3108 | adenosine 5′-diphosphate (ADP) | 1.3E−05 | 1.164E−05 | 0.7466 | 0.1627 | 0.2313 | 0.8626 | 0.0064 | 0.0157 | 0.7410 |
| 37058 | succinylcarnitine | 1.4E−05 | 1.198E−05 | 1.5749 | 0.7291 | 0.5540 | 0.9290 | 0.0152 | 0.0255 | 1.3840 |
| 37202 | 4-androsten-3beta,17beta-diol | 1.5E−05 | 1.242E−05 | 0.7759 | 0.2829 | 0.3309 | 1.3527 | 0.3546 | 0.1930 | 1.2828 |
| | disulfate 1 | | | | | | | | | |
| 1361 | pentadecanoate (15:0) | 1.6E−05 | 1.375E−05 | 0.8034 | 0.8579 | 0.6007 | 1.0474 | 0.5080 | 0.2524 | 0.9049 |
| 1301 | lysine | 2.2E−05 | 1.858E−05 | 1.5717 | 0.6977 | 0.5404 | 1.3324 | 0.2270 | 0.1416 | 1.4210 |
| 22171 | glycylproline | 3E−05 | 2.422E−05 | 1.4058 | 0.0120 | 0.0646 | 3.0403 | 0.0103 | 0.0205 | 2.8213 |
| 22175 | aspartylphenylalanine | 3E−05 | 2.426E−05 | 1.6947 | 0.0530 | 0.1278 | 2.6327 | 0.0072 | 0.0168 | 2.9412 |
| 32197 | 3-(4-hydroxyphenyl)lactate | 3.1E−05 | 2.481E−05 | 1.2467 | 0.0049 | 0.0441 | 1.8140 | 0.0241 | 0.0324 | 1.3510 |
| 35626 | 1-myristoylglycerophosphocholine | 3.2E−05 | 2.523E−05 | 2.3929 | 0.6642 | 0.5264 | 1.0678 | 0.2114 | 0.1369 | 1.2595 |
| 35627 | 1-myristoylglycerophospho- | 4.1E−05 | 3.197E−05 | 1.7902 | 0.9700 | 0.6302 | 1.0040 | 0.2896 | 0.1694 | 0.9431 |
| | ethanolamine | | | | | | | | | |
| 35428 | tiglyl carnitine | 4.7E−05 | 3.64E−05 | 1.5202 | 0.0149 | 0.0667 | 1.5850 | 0.3087 | 0.1770 | 1.5909 |
| 3155 | 3-ureidopropionate | 4.8E−05 | 3.694E−05 | 0.6847 | 0.6433 | 0.5167 | 1.1618 | 0.0858 | 0.0752 | 1.3666 |
| 32380 | nicotinamide adenine dinucleotide | 4.9E−05 | 3.694E−05 | 2.0890 | 0.0385 | 0.1120 | 0.6852 | 0.2570 | 0.1563 | 0.7039 |
| | phosphate (NADP+) | | | | | | | | | |
| 33449 | adenosine 5′-triphosphate (ATP) | 0.0001 | 4.416E−05 | 0.6983 | 0.1912 | 0.2538 | 0.7455 | 0.0000 | 0.0008 | 0.4480 |
| 32562 | pregnen-diol disulfate | 0.0001 | 0.0001 | 0.7838 | 0.3739 | 0.3876 | 1.0807 | 0.5852 | 0.2779 | 0.9883 |
| 37538 | 15-HETE | 0.0001 | 0.0001 | 1.5407 | 0.8138 | 0.5876 | 1.1126 | 0.2931 | 0.1701 | 1.1631 |
| 37083 | alanylproline | 0.0001 | 4.399E−05 | 1.5274 | 0.0801 | 0.1618 | 2.3264 | 0.0490 | 0.0499 | 2.0471 |
| 37093 | alanylleucine | 0.0002 | 0.0001 | 2.0969 | 0.4398 | 0.4227 | 1.1477 | 0.0235 | 0.0324 | 2.2294 |
| 31591 | androsterone sulfate | 0.0003 | 0.0002 | 0.7987 | 0.0455 | 0.1214 | 1.3709 | 0.1190 | 0.0930 | 1.2336 |
| 32980 | adrenate (22:4n6) | 0.0003 | 0.0002 | 1.2378 | 0.0236 | 0.0898 | 1.2236 | 0.0000 | 0.0008 | 1.4992 |
| 31609 | N1-methylguanosine | 0.0003 | 0.0002 | 1.2092 | 0.0113 | 0.0632 | 1.6745 | 0.0095 | 0.0195 | 1.4342 |
| 35128 | ketamine | 0.0003 | 0.0002 | 1.4679 | 0.6281 | 0.5084 | 1.1403 | 0.0421 | 0.0456 | 1.4483 |
| 35431 | 2-methylbutyroylcarnitine | 0.0003 | 0.0002 | 1.3277 | 0.0011 | 0.0251 | 1.5389 | 0.0686 | 0.0647 | 1.3283 |
| 37203 | 4-androsten-3beta,17beta- | 0.0004 | 0.0003 | 0.7788 | 0.4971 | 0.4470 | 1.0682 | 0.8235 | 0.3584 | 1.0066 |
| | dioldisulfate 2 | | | | | | | | | |
| 27716 | bilirubin (Z,Z) | 0.0006 | 0.0004 | 0.8097 | 0.9752 | 0.6302 | 0.9625 | 0.4850 | 0.2445 | 0.8558 |
| 34406 | valerylcarnitine | 0.0007 | 0.0004 | 1.2819 | 0.0542 | 0.1278 | 1.2441 | 0.0194 | 0.0295 | 1.4141 |
| 34398 | glycylleucine | 0.0008 | 0.0005 | 1.4343 | 0.0022 | 0.0281 | 2.5922 | 0.0012 | 0.0061 | 2.9299 |
| 37752 | 13-HODE + 9-HODE | 0.0011 | 0.0006 | 1.3264 | 0.2977 | 0.3378 | 1.2262 | 0.0270 | 0.0351 | 1.3782 |
| 15821 | fucose | 0.0011 | 0.0006 | 1.4559 | 0.4180 | 0.4147 | 1.1233 | 0.4588 | 0.2357 | 1.2518 |
| 34396 | choline phosphate | 0.0012 | 0.0007 | 1.6764 | 0.0285 | 0.0960 | 0.3963 | 0.0000 | 0.0002 | 0.0430 |
| 34418 | cytidine 5′-diphosphocholine | 0.0013 | 0.0007 | 1.2818 | 0.3645 | 0.3834 | 1.2992 | 0.3282 | 0.1846 | 1.1698 |
| 36602 | 1-oleoylglycerophosphoinositol | 0.0014 | 0.0008 | 1.3831 | 0.3366 | 0.3660 | 1.1374 | 0.0069 | 0.0164 | 1.3173 |
| 35628 | 1-oleoylglycerophospho- | 0.0016 | 0.0009 | 1.3535 | 0.5192 | 0.4561 | 1.0950 | 0.0192 | 0.0295 | 1.2800 |
| | ethanolamine | | | | | | | | | |
| 21188 | 1-stearoylglycerol (1-monostearin) | 0.0017 | 0.0009 | 1.3353 | 0.8483 | 0.5955 | 1.0815 | 0.0001 | 0.0019 | 1.6631 |
| 1118 | arachidate (20:0) | 0.0018 | 0.001 | 1.3959 | 0.7790 | 0.5723 | 1.0688 | 0.3320 | 0.1848 | 1.2144 |
| 21184 | 1-oleoylglycerol (1-monoolein) | 0.0019 | 0.001 | 1.4805 | 0.7232 | 0.5530 | 0.9054 | 0.2151 | 0.1377 | 1.2539 |
| 34656 | 2-arachidonoylglycerophospho- | 0.0024 | 0.0012 | 0.7781 | 0.5602 | 0.4712 | 0.9951 | 0.8264 | 0.3584 | 0.9278 |
| | ethanolamine | | | | | | | | | |
| 1589 | N-acetylmethionine | 0.0024 | 0.0012 | 1.3539 | 0.0884 | 0.1700 | 2.3971 | 0.0143 | 0.0251 | 2.3350 |
| 35687 | 2-oleoylglycerophospho- | 0.0027 | 0.0013 | 1.2656 | 0.5786 | 0.4819 | 1.1005 | 0.2050 | 0.1341 | 1.1747 |
| | ethanolamine | | | | | | | | | |
| 1561 | alpha-tocopherol | 0.0029 | 0.0014 | 1.1977 | 0.4378 | 0.4227 | 0.9839 | 0.2878 | 0.1688 | 1.0442 |
| 32672 | pyroglutamine | 0.0032 | 0.0016 | 0.9551 | 0.8632 | 0.6008 | 1.0450 | 0.4190 | 0.2214 | 0.7556 |
| 20714 | methyl-alpha-glucopyranoside | 0.0036 | 0.0017 | 1.5768 | 0.3013 | 0.3392 | 0.6076 | 0.3168 | 0.1801 | 1.1074 |
| 32379 | scyllo-inositol | 0.0038 | 0.0018 | 0.8927 | 0.9696 | 0.6302 | 0.9992 | 0.5025 | 0.2503 | 1.0929 |
| 32553 | phenol sulfate | 0.0038 | 0.0018 | 0.8015 | 0.6235 | 0.5059 | 1.1328 | 0.7288 | 0.3255 | 0.8787 |
| 31530 | threonylphenylalanine | 0.0038 | 0.0018 | 1.8909 | 0.5790 | 0.4819 | 1.1509 | 0.0305 | 0.0376 | 2.4724 |
| 1497 | ethanolamine | 0.0042 | 0.0019 | 1.2250 | 0.0055 | 0.0447 | 1.2915 | 0.0000 | 0.0011 | 1.5381 |
| 37478 | docosapentaenoate | 0.0045 | 0.0021 | 1.5229 | 0.0900 | 0.1711 | 1.2613 | 0.0057 | 0.0148 | 1.8681 |
| | (n6 DPA; 22:5n6) | | | | | | | | | |
| 32792 | andro steroid monosulfate 2 | 0.0048 | 0.0022 | 0.8343 | 0.1448 | 0.2198 | 1.1349 | 0.7907 | 0.3473 | 0.9667 |
| 18357 | glycylvaline | 0.0048 | 0.0022 | 1.2595 | 0.0489 | 0.1235 | 1.0407 | 0.0042 | 0.0133 | 1.4685 |
| 31260 | glucose-6-phosphate (G6P) | 0.005 | 0.0023 | 0.7327 | 0.9487 | 0.6302 | 0.8770 | 0.5594 | 0.2694 | 0.6543 |
| 18790 | acetylcholine | 0.0052 | 0.0024 | 0.8183 | 0.2137 | 0.2783 | 0.7852 | 0.0130 | 0.0243 | 0.6454 |
| 27447 | 1-linoleoylglycerol | 0.0053 | 0.0024 | 1.3016 | 0.1160 | 0.1941 | 1.2719 | 0.0834 | 0.0746 | 1.5063 |
| | (1-monolinolein) | | | | | | | | | |
| 35159 | cysteine-glutathione disulfide | 0.0053 | 0.0024 | 1.2938 | 0.0098 | 0.0586 | 1.7915 | 0.0079 | 0.0175 | 1.7349 |
| 33970 | cis-vaccenate (18:1n7) | 0.0054 | 0.0024 | 1.2878 | 0.8072 | 0.5858 | 1.0118 | 0.0607 | 0.0589 | 1.3005 |
| 35256 | 2-arachidonoylglycerophospho- | 0.0057 | 0.0025 | 0.7951 | 0.3157 | 0.3492 | 1.1561 | 0.5369 | 0.2629 | 0.9577 |
| | choline | | | | | | | | | |
| 17945 | 2-hydroxystearate | 0.0062 | 0.0027 | 1.3275 | 0.0081 | 0.0511 | 1.3578 | 0.0235 | 0.0324 | 1.3367 |
| 32807 | taurocholenate sulfate | 0.0064 | 0.0028 | 0.7875 | 0.1495 | 0.2236 | 1.1135 | 0.9729 | 0.3967 | 0.9418 |
| 36103 | p-cresol sulfate | 0.0067 | 0.003 | 0.7883 | 0.5740 | 0.4802 | 1.2395 | 0.9884 | 0.3998 | 0.9829 |
| 36738 | gamma-glutamylglutamate | 0.0078 | 0.0034 | 1.2069 | 0.0979 | 0.1771 | 1.7623 | 0.3395 | 0.1878 | 1.2287 |
| 27672 | 3-indoxyl sulfate | 0.0086 | 0.0038 | 0.4768 | 0.3137 | 0.3481 | 1.2350 | 0.3332 | 0.1848 | 1.2041 |
| 34585 | 4-hydroxybutyrate (GHB) | 0.0107 | 0.0046 | 1.4057 | 0.2391 | 0.2981 | 1.2594 | 0.0134 | 0.0243 | 1.9568 |
| 19503 | stearoyl sphingomyelin | 0.012 | 0.0051 | 0.8476 | 0.7864 | 0.5758 | 0.9210 | 0.3437 | 0.1894 | 0.9947 |
| 12102 | phosphoethabolamine | 0.0124 | 0.0053 | 1.4084 | 0.4304 | 0.4201 | 0.7657 | 0.0056 | 0.0148 | 0.1747 |
| 35186 | 1-arachidonoylglycerophospho- | 0.0126 | 0.0054 | 0.9210 | 0.1578 | 0.2285 | 1.1177 | 0.2446 | 0.1503 | 1.0836 |
| | ethanolamine | | | | | | | | | |
| 27727 | glutathione, oxidized (GSSG) | 0.0132 | 0.0055 | 0.9154 | 0.3395 | 0.3679 | 0.9189 | 0.3655 | 0.1983 | 0.8581 |
| 37418 | 1-pentadecanoylglycero- | 0.0144 | 0.006 | 1.4082 | 0.9776 | 0.6302 | 1.2763 | 0.1024 | 0.0841 | 1.6725 |
| | phosphocholine | | | | | | | | | |
| 35320 | catechol sulfate | 0.0145 | 0.006 | 0.5918 | 0.4747 | 0.4390 | 1.4079 | 0.8987 | 0.3793 | 0.8798 |
| 37190 | 5alpha-androstan-3beta,17beta-diol | 0.0152 | 0.0062 | 0.8095 | 0.2861 | 0.3323 | 1.3632 | 0.5289 | 0.2615 | 1.3012 |
| | disulfate | | | | | | | | | |
| 33935 | piperine | 0.02 | 0.008 | 1.1046 | 0.6552 | 0.5226 | 0.9456 | 0.3512 | 0.1917 | 1.1069 |
| 35631 | 1-palmitoylglycerophospho- | 0.0216 | 0.0085 | 1.1989 | 0.3337 | 0.3640 | 1.1561 | 0.0157 | 0.0261 | 1.2994 |
| | ethanolamine | | | | | | | | | |
| 12110 | isocitrate | 0.0221 | 0.0087 | 0.8190 | 0.7406 | 0.5588 | 0.9831 | 0.5637 | 0.2705 | 1.0695 |
| 34407 | isovalerylcarnitine | 0.0226 | 0.0089 | 1.3073 | 0.0089 | 0.0555 | 1.5247 | 0.1406 | 0.1031 | 1.3621 |
| 27738 | threonate | 0.0252 | 0.0098 | 0.5796 | 0.2981 | 0.3378 | 0.8809 | 0.1986 | 0.1315 | 1.1153 |
| 34258 | 2-docosahexaenoylglycero- | 0.0257 | 0.01 | 0.8530 | 0.4578 | 0.4310 | 0.9878 | 0.7711 | 0.3409 | 0.8962 |
| | phosphoethanolamine | | | | | | | | | |
| 32506 | 2-linoleoylglycerol | 0.0269 | 0.0104 | 1.2801 | 0.0293 | 0.0964 | 1.3141 | 0.0252 | 0.0335 | 1.4894 |
| | (2-monolinolein) | | | | | | | | | |
| 36808 | dimethylarginine | 0.0289 | 0.0111 | 1.2149 | 0.7786 | 0.5723 | 0.9359 | 0.9511 | 0.3901 | 0.9653 |
| | (SDMA + ADMA) | | | | | | | | | |
| 37496 | N-acetylputrescine | 0.0327 | 0.0123 | 0.7432 | 0.5030 | 0.4470 | 0.8853 | 0.0803 | 0.0731 | 1.3542 |
| 18369 | gamma-glutamylleucine | 0.0336 | 0.0126 | 1.2712 | 0.0649 | 0.1445 | 1.4672 | 0.0959 | 0.0801 | 1.2004 |
| 31787 | 3-carboxy-4-methyl-5-propyl-2- | 0.0363 | 0.0135 | 0.8852 | 0.9592 | 0.6302 | 0.9358 | 0.3935 | 0.2119 | 0.8851 |
| | furanpropanoate (CMPF) | | | | | | | | | |
| 37253 | 2-hydroxyglutarate | 0.0371 | 0.0138 | 4.3978 | 0.9989 | 0.6362 | 0.9446 | 0.4595 | 0.2357 | 0.5822 |
| 27718 | creatine | 0.0377 | 0.014 | 0.9427 | 0.9436 | 0.6302 | 0.9917 | 0.1831 | 0.1237 | 1.0718 |
| 12035 | pelargonate (9:0) | 0.0388 | 0.0143 | 1.0872 | 0.9872 | 0.6322 | 0.9928 | 0.0187 | 0.0292 | 0.8775 |
| 37070 | methylphosphate | 0.0411 | 0.015 | 1.0885 | 0.6486 | 0.5198 | 0.8520 | 0.1230 | 0.0943 | 1.1018 |
| 2849 | guanosine 5′-monophosphate | 0.0561 | 0.0199 | 1.1068 | 0.0430 | 0.1196 | 0.5421 | 0.0051 | 0.0141 | 0.5192 |
| | (GMP) | | | | | | | | | |
| 34214 | 1-arachidonoylglycero | 0.0598 | 0.021 | 1.0651 | 0.1326 | 0.2079 | 1.1316 | 0.0033 | 0.0121 | 1.2019 |
| | phosphoinositol | | | | | | | | | |
| 1585 | N-acetylalanine | 0.0793 | 0.0272 | 1.2058 | 0.0025 | 0.0289 | 2.3072 | 0.1328 | 0.0992 | 1.7737 |
| 34534 | laurylcarnitine | 0.0964 | 0.0323 | 1.5214 | 0.0810 | 0.1620 | 1.3196 | 0.0307 | 0.0376 | 1.4644 |
| 33961 | 1-stearoylglycerophosphocholine | 0.1053 | 0.0348 | 1.0164 | 0.0375 | 0.1110 | 1.2052 | 0.0432 | 0.0459 | 1.5333 |
| 32492 | caprylate (8:0) | 0.1139 | 0.0373 | 1.1163 | 0.9880 | 0.6322 | 1.0062 | 0.0125 | 0.0235 | 0.6925 |
| 35255 | 2-stearoylglycerophosphocholine | 0.133 | 0.043 | 1.0537 | 0.0539 | 0.1278 | 1.3310 | 0.0200 | 0.0300 | 1.6258 |
| 33441 | isobutyrylcarnitine | 0.1492 | 0.0475 | 0.9942 | 0.0048 | 0.0441 | 1.4825 | 0.1358 | 0.1007 | 1.3567 |
| 35855 | ribulose | 0.1684 | 0.0529 | 1.1928 | 0.0128 | 0.0646 | 2.2699 | 0.0136 | 0.0244 | 1.2616 |
| 33952 | myristoylcarnitine | 0.1965 | 0.0604 | 1.7507 | 0.0490 | 0.1235 | 1.2622 | 0.0201 | 0.0300 | 1.7254 |
| 33958 | glycyltyrosine | 0.2102 | 0.0639 | 1.2061 | 0.0106 | 0.0602 | 2.3342 | 0.1073 | 0.0875 | 2.2093 |
| 35688 | 2-palmitoylglycerophospho- | 0.2263 | 0.0673 | 1.1190 | 0.2626 | 0.3118 | 1.2662 | 0.0421 | 0.0456 | 1.2722 |
| | ethanolamine | | | | | | | | | |
| 34416 | 1-stearoylglycerophospho- | 0.2409 | 0.0711 | 1.0623 | 0.0494 | 0.1235 | 1.2114 | 0.0133 | 0.0243 | 1.4767 |
| | ethanolamine | | | | | | | | | |
| 35637 | cysteinylglycine | 0.266 | 0.0779 | 1.1549 | 0.3845 | 0.3942 | 0.9484 | 0.0360 | 0.0414 | 1.5609 |
| 35137 | N2,N2-dimethylguanosine | 0.2977 | 0.0854 | 0.8784 | 0.4907 | 0.4459 | 1.6160 | 0.0352 | 0.0412 | 1.2912 |
| 36761 | isoleucylisoleucine | 0.3175 | 0.0898 | 1.0246 | 0.1900 | 0.2535 | 0.7555 | 0.0021 | 0.0090 | 1.8901 |
| 35114 | 7-methylguanine | 0.3398 | 0.0946 | 0.9146 | 0.0540 | 0.1278 | 1.7909 | 0.0033 | 0.0122 | 1.2966 |
| 35675 | 2-hydroxypalmitate | 0.3815 | 0.1032 | 1.1376 | 0.0349 | 0.1064 | 1.2941 | 0.2574 | 0.1563 | 1.3723 |
| 33960 | 1-oleoylglycerophosphocholine | 0.4486 | 0.1183 | 1.1937 | 0.1699 | 0.2365 | 1.1700 | 0.0307 | 0.0376 | 1.6297 |
| 32342 | adenosine 5′-monophosphate | 0.6021 | 0.1507 | 0.7935 | 0.0191 | 0.0810 | 0.5291 | 0.0013 | 0.0067 | 0.4850 |
| | (AMP) | | | | | | | | | |
| 15335 | mannitol | 0.6702 | 0.1631 | 0.8962 | 0.1857 | 0.2488 | 1.4046 | 0.0207 | 0.0305 | 1.2965 |
| 33957 | 1-heptadecanoylglycero | 0.6734 | 0.1635 | 1.4504 | 0.0611 | 0.1370 | 1.3593 | 0.0120 | 0.0229 | 1.8571 |
| | phosphocholine | | | | | | | | | |
| 35160 | oleoylcarnitine | 0.6903 | 0.1672 | 1.3997 | 0.0145 | 0.0667 | 1.4870 | 0.0037 | 0.0127 | 2.0050 |
| 33477 | erythronate | 0.704 | 0.1694 | 0.9496 | 0.0587 | 0.1354 | 1.4643 | 0.0002 | 0.0021 | 1.3726 |
| 35127 | pro-hydroxy-pro | 0.7314 | 0.1745 | 0.9133 | 0.0877 | 0.1700 | 1.5127 | 0.0403 | 0.0451 | 1.1761 |
| 33871 | 1-eicosadienoylglycero- | 0.7961 | 0.1865 | 1.2787 | 0.0901 | 0.1711 | 1.2593 | 0.0110 | 0.0217 | 1.8045 |
| | phosphocholine | | | | | | | | | |
| 34409 | stearoylcarnitine | 0.9017 | 0.2057 | 1.5564 | 0.0258 | 0.0942 | 1.5893 | 0.0037 | 0.0127 | 2.1241 |
| 22189 | palmitoylcarnitine | 0.9084 | 0.2064 | 1.6256 | 0.0134 | 0.0657 | 1.4246 | 0.0089 | 0.0185 | 2.0203 |

TABLE 1B

| | | | | Ratio | | | Ratio | | | |
|---|---|---|---|---|---|---|---|---|---|---|
| | | | | Cancer | N_NOC | N_NOC | NOC/ | T_NOC | T_NOC | Ratio |
| | | | | Tumor/ | VS | VS | OC | VS | VS | T_NOC/ |
| Comp | | C VS T P- | C VS T | Control | N_OC P- | N_OC Q- | Adja- | T_OC P- | T_OC Q- | T_OC |
| ID | Name | VALUE | Q-VALUE | (T/C) | VALUE | VALUE | cent | VALUE | VALUE | Tumor |
| 15500 | carnitine | 1.4E−12 | 2.728E−11 | 1.2543 | 0.5028 | 0.4470 | 1.0387 | 0.0907 | 0.0780 | 1.0933 |
| 1898 | proline | 5.3E−12 | 8.588E−11 | 1.3923 | 0.0044 | 0.0441 | 1.2297 | 0.0020 | 0.0086 | 1.2368 |
| 54 | tryptophan | 2.9E−11 | 2.349E−10 | 1.2512 | 0.0047 | 0.0441 | 1.2270 | 0.0001 | 0.0018 | 1.2947 |
| 32975 | taurine | 1.4E−10 | 7.779E−10 | 0.6409 | 0.9504 | 0.6302 | 1.0364 | 0.1102 | 0.0883 | 0.8222 |
| 1284 | threonine | 1.9E−10 | 1.035E−09 | 1.3993 | 0.0597 | 0.1357 | 1.1837 | 0.0058 | 0.0149 | 1.2350 |
| 606 | uridine | 3E−10 | 1.421E−09 | 1.3379 | 0.1055 | 0.1852 | 1.1128 | 0.0010 | 0.0059 | 1.2784 |
| 60 | leucine | 4.7E−10 | 1.897E−09 | 1.2454 | 0.0003 | 0.0155 | 1.3605 | 0.0002 | 0.0024 | 1.3898 |
| 6146 | 2-aminoadipate | 5.3E−10 | 2.085E−09 | 1.6525 | 0.3913 | 0.3961 | 0.8972 | 0.3144 | 0.1794 | 1.0246 |
| 1359 | oleate (18:1n9) | 8.1E−10 | 2.959E−09 | 1.4134 | 0.7704 | 0.5721 | 1.0609 | 0.0049 | 0.0141 | 1.3210 |
| 1419 | 5-methylthioadenosine (MTA) | 2.1E−09 | 6.647E−09 | 1.5658 | 0.9395 | 0.6302 | 1.0373 | 0.2711 | 0.1613 | 1.1081 |
| 64 | phenylalanine | 2.9E−09 | 8.43E−09 | 1.2459 | 0.0029 | 0.0318 | 1.4145 | 0.0016 | 0.0076 | 1.4104 |
| 1299 | tyrosine | 4.3E−09 | 1.11E−08 | 1.2343 | 0.0038 | 0.0392 | 1.5168 | 0.0028 | 0.0108 | 1.4687 |
| 11777 | glycine | 4.7E−09 | 1.162E−08 | 1.3676 | 0.0340 | 0.1064 | 1.1579 | 0.0299 | 0.0376 | 1.1764 |
| 1105 | linoleate (18:2n6) | 1E−08 | 2.266E−08 | 1.4084 | 0.0008 | 0.0204 | 1.4241 | 0.0010 | 0.0057 | 1.4434 |
| 513 | creatinine | 1.2E−08 | 2.573E−08 | 0.7005 | 0.5418 | 0.4666 | 1.2272 | 0.6213 | 0.2896 | 0.9873 |
| 2766 | N-acetylgalactosamine | 1.3E−08 | 2.723E−08 | 2.0376 | 0.4920 | 0.4459 | 1.3785 | 0.2991 | 0.1719 | 1.3636 |
| 1494 | 5-oxoproline | 3.2E−08 | 5.87E−08 | 1.3941 | 0.0151 | 0.0670 | 1.6118 | 0.0572 | 0.0560 | 1.3254 |
| 605 | uracil | 3.9E−08 | 6.966E−08 | 1.8625 | 0.0006 | 0.0204 | 1.8463 | 0.0003 | 0.0027 | 2.0160 |
| 15365 | glycerol 3-phosphate (G3P) | 6.5E−08 | 1.093E−07 | 1.4659 | 0.1355 | 0.2103 | 0.8200 | 0.8962 | 0.3790 | 0.9890 |
| 35661 | lidocaine | 6.6E−08 | 1.102E−07 | 1.6411 | 0.2454 | 0.3014 | 1.4764 | 0.0148 | 0.0254 | 1.9789 |
| 3127 | hypoxanthine | 8.4E−08 | 1.378E−07 | 1.3214 | 0.0000 | 0.0028 | 1.5438 | 0.0003 | 0.0028 | 1.3975 |
| 15990 | glycerophosphorylcholine (GPC) | 8.5E−08 | 1.378E−07 | 1.5443 | 0.3578 | 0.3800 | 0.9399 | 0.5657 | 0.2705 | 1.0659 |
| 15136 | xanthosine | 9.1E−08 | 1.437E−07 | 1.9673 | 0.0324 | 0.1027 | 1.5805 | 0.0415 | 0.0456 | 1.3590 |
| 15948 | S-adenosylhomocysteine (SAH) | 9.9E−08 | 1.517E−07 | 1.2312 | 0.0007 | 0.0204 | 1.3262 | 0.0082 | 0.0175 | 1.2211 |
| 31453 | cysteine | 1.3E−07 | 1.851E−07 | 1.9429 | 0.0066 | 0.0499 | 1.3016 | 0.0025 | 0.0102 | 1.7826 |
| 15096 | N-acetylglucosamine | 1.4E−07 | 2.019E−07 | 2.4319 | 0.1096 | 0.1908 | 1.8005 | 0.0239 | 0.0324 | 1.7337 |
| 33447 | palmitoleate (16:1n7) | 4E−07 | 5.071E−07 | 1.2929 | 0.0466 | 0.1223 | 1.2014 | 0.1757 | 0.1203 | 1.1595 |
| 1649 | valine | 4.3E−07 | 5.377E−07 | 1.1475 | 0.0014 | 0.0262 | 1.2464 | 0.0007 | 0.0044 | 1.2735 |
| 554 | adenine | 4.7E−07 | 5.79E−07 | 1.4385 | 0.3697 | 0.3864 | 1.0740 | 0.0587 | 0.0572 | 1.1661 |
| 1508 | pantothenate | 4.8E−07 | 5.834E−07 | 1.1803 | 0.0303 | 0.0983 | 1.3433 | 0.0062 | 0.0156 | 1.3840 |
| 1302 | methionine | 5.8E−07 | 6.937E−07 | 1.2140 | 0.0211 | 0.0850 | 1.6127 | 0.0569 | 0.0560 | 1.5274 |
| 1648 | serine | 1.4E−06 | 1.517E−06 | 1.3310 | 0.0919 | 0.1716 | 1.2496 | 0.0144 | 0.0251 | 1.3279 |
| 1493 | ornithine | 2.4E−06 | 2.597E−06 | 1.5806 | 0.8271 | 0.5903 | 1.2284 | 0.5654 | 0.2705 | 1.1409 |
| 1125 | isoleucine | 2.6E−06 | 2.795E−06 | 1.1602 | 0.0002 | 0.0149 | 1.4599 | 0.0006 | 0.0044 | 1.3873 |
| 59 | histidine | 3.1E−06 | 3.233E−06 | 1.1863 | 0.0098 | 0.0586 | 1.1381 | 0.0075 | 0.0171 | 1.1625 |
| 1303 | malate | 3.2E−06 | 3.314E−06 | 1.4488 | 0.0692 | 0.1490 | 1.1460 | 0.0050 | 0.0141 | 1.3388 |
| 1126 | alanine | 3.4E−06 | 3.473E−06 | 1.3058 | 0.1843 | 0.2479 | 1.1000 | 0.0112 | 0.0218 | 1.1653 |
| 1604 | urate | 5E−06 | 4.861E−06 | 0.8080 | 0.1322 | 0.2079 | 1.1180 | 0.9965 | 0.4023 | 0.9742 |
| 1336 | palmitate (16:0) | 6.2E−06 | 5.928E−06 | 1.1014 | 0.0023 | 0.0281 | 1.1677 | 0.0051 | 0.0141 | 1.1558 |
| 514 | cytidine | 7.2E−06 | 6.756E−06 | 1.5003 | 0.0248 | 0.0920 | 0.6040 | 0.8239 | 0.3584 | 1.1129 |
| 1444 | pipecolate | 1E−05 | 9.154E−06 | 1.2978 | 0.2277 | 0.2884 | 1.2135 | 0.0307 | 0.0376 | 1.2782 |
| 1110 | arachidonate (20:4n6) | 1.1E−05 | 9.775E−06 | 1.2443 | 0.0669 | 0.1459 | 1.1853 | 0.0022 | 0.0091 | 1.2709 |
| 15996 | aspartate | 3E−05 | 2.422E−05 | 1.2468 | 0.1315 | 0.2079 | 1.1669 | 0.2381 | 0.1472 | 1.1181 |
| 1558 | 4-acetamidobutanoate | 0.0001 | 4.178E−05 | 0.7334 | 0.8365 | 0.5917 | 1.2146 | 0.5996 | 0.2820 | 1.0557 |
| 32425 | dehydroisoandrosterone | 0.0001 | 0.0001 | 0.8013 | 0.3882 | 0.3955 | 1.0169 | 0.6068 | 0.2842 | 0.9558 |
| | sulfate (DHEA-S) | | | | | | | | | |
| 1366 | trans-4-hydroxyproline | 0.0001 | 0.0001 | 1.2889 | 0.3749 | 0.3876 | 0.8460 | 0.8738 | 0.3726 | 0.9048 |
| 12083 | ribose | 0.0001 | 0.0001 | 1.3406 | 0.0015 | 0.0262 | 1.8538 | 0.0002 | 0.0021 | 1.8022 |
| 15915 | S-adenosylmethionine (SAM) | 0.0001 | 0.0001 | 1.5259 | 0.9543 | 0.6302 | 1.0203 | 0.0765 | 0.0702 | 1.1843 |
| 11398 | asparagine | 0.0001 | 0.0001 | 1.4370 | 0.0125 | 0.0646 | 1.3092 | 0.0958 | 0.0801 | 1.2640 |
| 22185 | N-acetylaspartate (NAA) | 0.0001 | 0.0001 | 1.5287 | 0.0495 | 0.1235 | 1.2414 | 0.0650 | 0.0622 | 1.1846 |
| 1592 | N-acetylneuraminate | 0.0001 | 0.0001 | 1.8207 | 0.8843 | 0.6115 | 1.0967 | 0.3229 | 0.1825 | 1.0969 |
| 53 | glutamine | 0.0002 | 0.0002 | 1.1291 | 0.7241 | 0.5530 | 1.0155 | 0.0183 | 0.0287 | 1.1182 |
| 19934 | myo-inositol | 0.0003 | 0.0002 | 0.9093 | 0.9886 | 0.6322 | 0.9898 | 0.1048 | 0.0858 | 1.0898 |
| 36984 | Isobar: fructose 1,6-diphosphate, | 0.0004 | 0.0002 | 0.6610 | 0.5238 | 0.4572 | 0.7740 | 0.0008 | 0.0051 | 0.3565 |
| | glucose 1,6-diphosphate | | | | | | | | | |
| 4966 | xylitol | 0.0004 | 0.0002 | 1.2413 | 0.0290 | 0.0964 | 1.5918 | 0.0001 | 0.0019 | 1.8129 |
| 1559 | 5,6-dihydrouracil | 0.0005 | 0.0003 | 1.4286 | 0.0051 | 0.0441 | 1.5557 | 0.0073 | 0.0171 | 1.4564 |
| 35133 | N2-methylguanosine | 0.0007 | 0.0004 | 1.2336 | 0.2199 | 0.2840 | 1.2910 | 0.0758 | 0.0698 | 1.2182 |
| 1827 | riboflavin (Vitamin B2) | 0.0007 | 0.0004 | 1.2503 | 0.0482 | 0.1235 | 1.5993 | 0.0259 | 0.0342 | 1.5053 |
| 2132 | citrulline | 0.0008 | 0.0004 | 1.3540 | 0.4104 | 0.4103 | 0.9233 | 0.3324 | 0.1848 | 0.8118 |
| 57 | glutamate | 0.0011 | 0.0006 | 1.0944 | 0.1128 | 0.1909 | 1.0538 | 0.0209 | 0.0305 | 1.1244 |
| 1365 | myristate (14:0) | 0.0011 | 0.0006 | 1.0944 | 0.4987 | 0.4470 | 1.0207 | 0.2218 | 0.1401 | 0.9406 |
| 2856 | uridine 5′-monophosphate (UMP) | 0.0014 | 0.0008 | 0.7520 | 0.0071 | 0.0510 | 0.4653 | 0.0003 | 0.0027 | 0.2778 |
| 37059 | malonylcarnitine | 0.0016 | 0.0009 | 1.3228 | 0.1931 | 0.2546 | 1.1975 | 0.2659 | 0.1587 | 1.1999 |
| 1516 | sarcosine (N-Methylglycine) | 0.0018 | 0.001 | 1.6614 | 0.1669 | 0.2344 | 1.0950 | 0.2567 | 0.1563 | 1.0535 |
| 1643 | fumarate | 0.0019 | 0.001 | 1.3148 | 0.0120 | 0.0646 | 1.2963 | 0.6971 | 0.3190 | 0.9504 |
| 2372 | cytidine 5′-monophosphate | 0.0021 | 0.0011 | 1.1698 | 0.4186 | 0.4147 | 0.8668 | 0.0939 | 0.0801 | 0.8575 |
| | (5′-CMP) | | | | | | | | | |
| 527 | lactate | 0.0022 | 0.0011 | 1.0960 | 0.2045 | 0.2673 | 1.1001 | 0.1096 | 0.0883 | 1.1083 |
| 1437 | succinate | 0.0025 | 0.0013 | 1.2840 | 0.8469 | 0.5955 | 1.0776 | 0.0282 | 0.0364 | 1.3244 |
| 1566 | 3-aminoisobutyrate | 0.0026 | 0.0013 | 1.2252 | 0.5469 | 0.4677 | 1.9943 | 0.7731 | 0.3410 | 0.8360 |
| 15122 | glycerol | 0.0029 | 0.0014 | 1.1375 | 0.0012 | 0.0251 | 1.2338 | 0.0001 | 0.0016 | 1.3959 |
| 1121 | margarate (17:0) | 0.009 | 0.0039 | 1.1160 | 0.0025 | 0.0289 | 1.1720 | 0.0469 | 0.0483 | 1.1391 |
| 12055 | galactose | 0.0096 | 0.0042 | 1.2630 | 0.0187 | 0.0805 | 1.2559 | 0.0006 | 0.0044 | 1.4373 |
| 5278 | nicotinamide adenine | 0.0131 | 0.0055 | 1.7379 | 0.0203 | 0.0832 | 0.5728 | 0.1203 | 0.0933 | 0.7146 |
| | dinucleotide (NAD+) | | | | | | | | | |
| 15140 | kynurenine | 0.0134 | 0.0056 | 1.3670 | 0.0264 | 0.0942 | 1.6187 | 0.2937 | 0.1701 | 1.0544 |
| 32328 | hexanoylcarnitine | 0.0188 | 0.0076 | 1.2092 | 0.2526 | 0.3053 | 1.1399 | 0.0425 | 0.0456 | 1.3009 |
| 1574 | histamine | 0.0191 | 0.0077 | 1.1915 | 0.2213 | 0.2847 | 0.9140 | 0.8185 | 0.3580 | 0.9509 |
| 1572 | glycerate | 0.0288 | 0.0111 | 1.0776 | 0.0073 | 0.0510 | 2.0098 | 0.0067 | 0.0162 | 1.9263 |
| 11438 | phosphate | 0.0323 | 0.0122 | 1.1204 | 0.3605 | 0.3816 | 1.1112 | 0.4569 | 0.2356 | 1.0013 |
| 63 | cholesterol | 0.0401 | 0.0147 | 1.0525 | 0.6863 | 0.5367 | 1.0132 | 0.6003 | 0.2820 | 0.9845 |
| 15753 | hippurate | 0.043 | 0.0156 | 0.3444 | 0.3429 | 0.3691 | 2.7235 | 0.4425 | 0.2305 | 2.1170 |
| 15053 | sorbitol | 0.0513 | 0.0185 | 1.3776 | 0.3475 | 0.3703 | 1.0848 | 0.0053 | 0.0144 | 1.4424 |
| 590 | hypotaurine | 0.0541 | 0.0193 | 1.1282 | 0.8236 | 0.5903 | 0.9378 | 0.9161 | 0.3806 | 1.0150 |
| 37506 | palmitoyl sphingomyelin | 0.0544 | 0.0194 | 1.0672 | 0.1479 | 0.2221 | 0.9104 | 0.6280 | 0.2921 | 1.0152 |
| 35153 | 1-docosahexaenoylglycerol (1- | 0.0572 | 0.0203 | 1.3518 | 0.7668 | 0.5719 | 0.9644 | 0.1369 | 0.1012 | 1.2366 |
| | monodocosahexaenoin) | | | | | | | | | |
| 594 | nicotinamide | 0.0625 | 0.0219 | 1.0741 | 0.0791 | 0.1606 | 1.0855 | 0.0074 | 0.0171 | 1.1850 |
| 27743 | triethyleneglycol | 0.0642 | 0.0225 | 0.9022 | 0.7707 | 0.5721 | 0.9372 | 0.4665 | 0.2387 | 0.9358 |
| 32418 | myristoleate (14:1n5) | 0.0967 | 0.0323 | 1.1350 | 0.0513 | 0.1264 | 1.1489 | 0.5749 | 0.2743 | 0.9037 |
| 1414 | 3-phosphoglycerate | 0.0982 | 0.0327 | 0.7285 | 0.9125 | 0.6222 | 1.0783 | 0.1583 | 0.1127 | 0.5864 |
| 33936 | octanoylcarnitine | 0.0987 | 0.0328 | 0.9296 | 0.7781 | 0.5723 | 1.0674 | 0.5247 | 0.2600 | 1.1034 |
| 35665 | N-acetyl-aspartyl-glutamate | 0.11 | 0.0363 | 1.1213 | 0.1111 | 0.1909 | 1.1907 | 0.2628 | 0.1574 | 1.1697 |
| | (NAAG) | | | | | | | | | |
| 34592 | ophthalmate | 0.1109 | 0.0364 | 0.9763 | 0.6946 | 0.5404 | 0.9727 | 0.9168 | 0.3806 | 1.0580 |
| 36776 | 7-alpha-hydroxy-3-oxo-4- | 0.1255 | 0.0408 | 1.1101 | 0.3455 | 0.3696 | 1.1728 | 0.5488 | 0.2674 | 0.8501 |
| | cholestenoate (7-Hoca) | | | | | | | | | |
| 35253 | 2-palmitoylglycerophosphocholine | 0.1263 | 0.0409 | 1.0446 | 0.0884 | 0.1700 | 1.2786 | 0.1172 | 0.0928 | 1.7129 |
| 33230 | 1-palmitoleoylglycero- | 0.1358 | 0.0437 | 1.5581 | 0.4553 | 0.4299 | 1.1682 | 0.0742 | 0.0693 | 1.4246 |
| | phosphocholine | | | | | | | | | |
| 32675 | C-glycosyltryptophan | 0.1373 | 0.0441 | 1.1181 | 0.3456 | 0.3696 | 1.0446 | 0.2867 | 0.1686 | 1.0904 |
| 35638 | xylonate | 0.1576 | 0.0498 | 0.8350 | 0.5519 | 0.4690 | 1.0363 | 0.1676 | 0.1171 | 1.2666 |
| 34875 | 2-docosapentaenoylglycero- | 0.1576 | 0.0498 | 0.8239 | 0.4444 | 0.4246 | 0.9173 | 0.7033 | 0.3199 | 0.7928 |
| | phosphoethanolamine | | | | | | | | | |
| 15496 | agmatine | 0.1632 | 0.0514 | 1.0578 | 0.5715 | 0.4793 | 2.2947 | 0.1774 | 0.1211 | 1.3248 |
| 1358 | stearate (18:0) | 0.1712 | 0.0536 | 1.0409 | 0.0045 | 0.0441 | 1.1534 | 0.0008 | 0.0053 | 1.2131 |
| 18371 | GDP-mannose | 0.1776 | 0.0553 | 1.1528 | 0.8376 | 0.5917 | 0.9405 | 0.4462 | 0.2312 | 1.0278 |
| 35884 | 2-eicosatrienoylglycero- | 0.1794 | 0.0556 | 1.0390 | 0.3172 | 0.3496 | 1.5740 | 0.2154 | 0.1377 | 1.4313 |
| | phosphocholine | | | | | | | | | |
| 2342 | serotonin (5HT) | 0.2002 | 0.0612 | 0.8725 | 0.7476 | 0.5607 | 0.8934 | 0.5892 | 0.2792 | 1.0478 |
| 33955 | 1-palmitoylglycerophosphocholine | 0.2237 | 0.0672 | 1.0422 | 0.0665 | 0.1459 | 1.1209 | 0.0636 | 0.0611 | 1.3890 |
| 35257 | 2-linoleoylglycerophosphocholine | 0.2247 | 0.0672 | 1.0362 | 0.1207 | 0.1999 | 1.2879 | 0.1919 | 0.1286 | 1.2142 |
| 20488 | glucose | 0.2257 | 0.0673 | 0.8800 | 0.2625 | 0.3118 | 0.8116 | 0.3295 | 0.1847 | 1.1816 |
| 2730 | gamma-glutamylglutamine | 0.2538 | 0.0745 | 1.1874 | 0.6071 | 0.4989 | 0.9534 | 0.8343 | 0.3610 | 0.7011 |
| 485 | spermidine | 0.2714 | 0.0788 | 1.6646 | 0.1527 | 0.2263 | 0.7318 | 0.2606 | 0.1573 | 1.3201 |
| 32394 | pyroglutamylvaline | 0.2724 | 0.0788 | 0.9394 | 0.5280 | 0.4582 | 0.6961 | 0.0855 | 0.0752 | 1.4176 |
| 1573 | guanosine | 0.2856 | 0.0824 | 0.9741 | 0.9621 | 0.6302 | 0.9953 | 0.1314 | 0.0990 | 1.0567 |
| 15488 | acetylphosphate | 0.2907 | 0.0836 | 1.0627 | 0.2663 | 0.3150 | 0.9202 | 0.2037 | 0.1341 | 0.8871 |
| 35126 | phenylacetylglutamine | 0.3003 | 0.086 | 0.3934 | 0.2904 | 0.3349 | 2.2048 | 0.2243 | 0.1408 | 1.8656 |
| 34410 | cytidine-5′-diphosphoethanolamine | 0.3077 | 0.0879 | 1.0042 | 0.4929 | 0.4459 | 0.9996 | 0.2148 | 0.1377 | 0.8323 |
| 34419 | 1-linoleoylglycerophosphocholine | 0.3093 | 0.0881 | 1.0467 | 0.3624 | 0.3825 | 1.2830 | 0.1375 | 0.1012 | 1.5680 |
| 15705 | cystathionine | 0.3259 | 0.0917 | 1.1588 | 0.1648 | 0.2324 | 0.8484 | 0.1815 | 0.1230 | 1.2588 |
| 542 | 3-hydroxybutyrate (BHBA) | 0.335 | 0.0938 | 1.0394 | 0.6978 | 0.5404 | 1.3721 | 0.8846 | 0.3764 | 1.3159 |
| 55 | beta-alanine | 0.3465 | 0.0961 | 0.9366 | 0.2596 | 0.3105 | 1.2564 | 0.9808 | 0.3983 | 0.8974 |
| 569 | caffeine | 0.3492 | 0.0963 | 0.9603 | 0.9852 | 0.6322 | 1.3570 | 0.7264 | 0.3255 | 1.3668 |
| 37475 | 4-acetaminophen sulfate | 0.3594 | 0.0982 | 0.8691 | 0.4377 | 0.4227 | 1.1959 | 0.7216 | 0.3253 | 1.3768 |
| 33420 | gamma-tocopherol | 0.3751 | 0.102 | 1.0016 | 0.1585 | 0.2285 | 1.4161 | 0.2057 | 0.1341 | 0.8640 |
| 17747 | sphingosine | 0.3771 | 0.1023 | 1.3711 | 0.2878 | 0.3331 | 1.0895 | 0.1104 | 0.0883 | 1.3760 |
| 15650 | N1-methyladenosine | 0.3855 | 0.1038 | 1.0138 | 0.1452 | 0.2198 | 1.1624 | 0.0049 | 0.0141 | 1.1902 |
| 599 | pyruvate | 0.3873 | 0.1039 | 1.1099 | 0.5588 | 0.4712 | 1.1298 | 0.2401 | 0.1480 | 0.8845 |
| 35819 | 2-palmitoleoylglycero- | 0.407 | 0.1086 | 1.1112 | 0.6094 | 0.4992 | 1.0559 | 0.5325 | 0.2620 | 0.9220 |
| | phosphocholine | | | | | | | | | |
| 587 | gluconate | 0.4457 | 0.1178 | 0.8638 | 0.1217 | 0.2004 | 0.7099 | 0.2239 | 0.1408 | 0.8142 |
| 35174 | mead acid (20:3n9) | 0.4507 | 0.1186 | 1.6095 | 0.4894 | 0.4459 | 0.7850 | 0.3147 | 0.1794 | 0.7945 |
| 577 | fructose | 0.4691 | 0.1228 | 1.0157 | 0.4277 | 0.4198 | 1.0615 | 0.0045 | 0.0140 | 1.2793 |
| 584 | mannose | 0.4831 | 0.1256 | 1.0744 | 0.8389 | 0.5917 | 0.9744 | 0.0037 | 0.0127 | 1.2834 |
| 15806 | maltose | 0.5027 | 0.1301 | 1.0284 | 0.1129 | 0.1909 | 1.2530 | 0.4100 | 0.2174 | 1.4206 |
| 18392 | theobromine | 0.5097 | 0.1316 | 0.9932 | 0.8970 | 0.6150 | 1.2033 | 0.9859 | 0.3996 | 1.2210 |
| 1416 | gamma-aminobutyrate (GABA) | 0.5183 | 0.1332 | 0.9446 | 0.1783 | 0.2430 | 1.3029 | 0.1174 | 0.0928 | 1.4597 |
| 32352 | guanine | 0.548 | 0.1384 | 1.0322 | 0.0005 | 0.0204 | 1.3769 | 0.2783 | 0.1651 | 1.2774 |
| 35623 | 1-arachidoylglycerophosphocholine | 0.5483 | 0.1384 | 1.1119 | 0.9773 | 0.6302 | 0.9718 | 0.2587 | 0.1566 | 1.4464 |
| 1564 | citrate | 0.5553 | 0.1399 | 1.0084 | 0.3903 | 0.3961 | 0.8374 | 0.0949 | 0.0801 | 1.0972 |
| 33442 | pseudouridine | 0.5749 | 0.1445 | 0.8560 | 0.4023 | 0.4047 | 1.2958 | 0.0629 | 0.0608 | 1.1218 |
| 37063 | gamma-glutamylalanine | 0.5844 | 0.1466 | 1.2530 | 0.3037 | 0.3394 | 1.0456 | 0.1923 | 0.1286 | 0.6337 |
| 555 | adenosine | 0.6033 | 0.1507 | 0.9109 | 0.0020 | 0.0281 | 0.2716 | 0.0014 | 0.0069 | 0.3267 |
| 1642 | caprate (10:0) | 0.6071 | 0.1513 | 1.0349 | 0.6324 | 0.5105 | 0.9736 | 0.1426 | 0.1042 | 0.9005 |
| 2127 | glutathione, reduced (GSH) | 0.6168 | 0.1531 | 1.0221 | 0.2153 | 0.2792 | 0.9604 | 0.9483 | 0.3900 | 1.1482 |
| 20675 | 1,5-anhydroglucitol (1,5-AG) | 0.6212 | 0.1538 | 0.9881 | 0.4121 | 0.4108 | 0.9482 | 0.9027 | 0.3802 | 1.0704 |
| 3147 | xanthine | 0.628 | 0.1551 | 1.2651 | 0.0283 | 0.0960 | 1.2086 | 0.4887 | 0.2455 | 1.2618 |
| 35254 | 2-oleoylglycerophosphocholine | 0.6345 | 0.1564 | 1.2696 | 0.0781 | 0.1596 | 1.3242 | 0.1315 | 0.0990 | 1.3567 |
| 603 | spermine | 0.6612 | 0.1622 | 1.0424 | 0.0993 | 0.1771 | 0.6612 | 0.2365 | 0.1467 | 0.8200 |
| 15877 | maltotriose | 0.6697 | 0.1631 | 1.2089 | 0.2341 | 0.2930 | 1.1637 | 0.9571 | 0.3918 | 1.1865 |
| 1123 | inosine | 0.6703 | 0.1631 | 1.0061 | 0.1627 | 0.2313 | 1.0762 | 0.0021 | 0.0090 | 1.1462 |
| 33937 | alpha-hydroxyisovalerate | 0.6941 | 0.1674 | 1.0000 | 0.0748 | 0.1578 | 1.2299 | 0.9100 | 0.3806 | 1.1201 |
| 1670 | urea | 0.7166 | 0.1721 | 1.0283 | 0.0127 | 0.0646 | 1.1853 | 0.0235 | 0.0324 | 1.2021 |
| 1481 | inositol 1-phosphate (I1P) | 0.7226 | 0.1732 | 1.0016 | 0.2534 | 0.3053 | 0.8317 | 0.1555 | 0.1117 | 0.8076 |
| 19266 | 2-arachidonoyl glycerol | 0.756 | 0.1797 | 1.0469 | 0.9683 | 0.6302 | 0.9004 | 0.1203 | 0.0933 | 1.2179 |
| 1645 | laurate (12:0) | 0.7578 | 0.1797 | 1.0118 | 0.0874 | 0.1700 | 0.9217 | 0.0001 | 0.0019 | 0.8124 |
| 34397 | 1-arachidonylglycerol | 0.7603 | 0.1799 | 1.0623 | 0.2549 | 0.3060 | 0.8914 | 0.9292 | 0.3850 | 0.9237 |
| 15910 | maltotetraose | 0.7886 | 0.1854 | 1.0561 | 0.8152 | 0.5876 | 0.9876 | 0.9485 | 0.3900 | 1.1374 |
| 37060 | methylglutaroylcarnitine | 0.7984 | 0.1866 | 0.6899 | 0.0972 | 0.1771 | 2.7956 | 0.0936 | 0.0801 | 1.6467 |
| 12025 | cis-aconitate | 0.8028 | 0.1873 | 0.9883 | 0.6763 | 0.5329 | 0.9026 | 0.2627 | 0.1574 | 1.0276 |
| 1640 | ascorbate (Vitamin C) | 0.821 | 0.1911 | 1.0018 | 0.8119 | 0.5876 | 1.0169 | 0.2942 | 0.1701 | 1.1529 |
| 558 | adenosine 5′diphosphoribose | 0.8463 | 0.1962 | 0.9337 | 0.1841 | 0.2479 | 0.7385 | 0.7111 | 0.3220 | 0.9482 |
| 33173 | 2-hydroxyacetaminophen sulfate | 0.8555 | 0.1979 | 0.6505 | 0.4525 | 0.4293 | 1.4420 | 0.6008 | 0.2820 | 1.1797 |
| 1408 | putrescine | 0.884 | 0.2025 | 1.0554 | 0.3823 | 0.3932 | 0.8668 | 0.4838 | 0.2445 | 1.0544 |
| 33821 | 1-eicosatrienoylglycero- | 0.904 | 0.2058 | 0.9828 | 0.2406 | 0.2987 | 1.5043 | 0.0562 | 0.0558 | 1.5172 |
| | phosphocholine | | | | | | | | | |
| 27665 | 1-methylnicotinamide | 0.9469 | 0.2134 | 0.9365 | 0.8594 | 0.6007 | 1.0928 | 0.0951 | 0.0801 | 1.1174 |
| 21044 | 2-hydroxybutyrate (AHB) | 0.9665 | 0.2174 | 1.0117 | 0.0058 | 0.0454 | 1.2906 | 0.0686 | 0.0647 | 1.2024 |
| 20699 | erythritol | 0.9684 | 0.2174 | 0.9460 | 0.0982 | 0.1771 | 1.2939 | 0.0180 | 0.0286 | 1.2313 |

To summarize the results in Tables 1A and 1B, 315 biomarkers were identified. Of these, 206 biomarkers were statistically significantly different between tumors (T) and non-cancer tissue adjacent to tumors (C), 131 biomarkers were identified as significantly different between high aggressive tumors (T_NOC) and less aggressive tumors (T_OC), and 86 biomarkers were identified as significantly different between non-cancer tissue adjacent to high aggressive cancer tumors (N_NOC) and non-cancer tissue adjacent to less aggressive cancer tumors (N_OC). Of the biomarkers that are statistically significantly changed in tumors that are high aggressive cancer (T_NOC) compared to tumors that are less aggressive cancer (T_OC) 34 biomarkers increase or decrease 10%-30%, 49 biomarkers increase or decrease 30%-50%, 37 biomarkers increase or decrease 50%-100% and 12 biomarkers increase or decrease >100%. The range of percent change is 10%-239%. The False Discovery Rate was less than or equal to 5% (i.e., q≦0.05).

### Example 2

### Random Forest Analysis for the Classification of Tissue Samples

The data obtained in Example 1 concerning the tissue samples was used to create a Random Forest model. Random Forest Analysis was carried out on the data obtained from tissue samples in Example 1 to classify them as Control, non-cancer tissue (C), Organ Confined Tumor (T_OC) (i.e. lower aggressive) or Non-Organ Confined Tumor (T_NOC) (i.e. high aggressive cancer).

It was found that 83% (Table 2) accuracy was achieved by Random Forest Classification of Non-cancer, control tissue compared to organ confined tumor tissue. A list of identified biomarker compounds that effectively separate the groups are presented in Tables 3A and 3B.

TABLE 2
Random Forest Classification of Cancer (Tumor)
vs. Non-cancer (Control) Tissue.

Predicted

| | | Control | Tumor | class. error |
|---|---|---|---|---|
| Actual | Control | 59 | 12 | 0.17 |
| | Tumor | 13 | 60 | 0.18 |

OOB error = 17%

The diagnostic parameters based on the Random Forest Analysis are that the Accuracy=83%; the Sensitivity=82, the Specificity=83, the Positive Predictive Value (PPV)=83, the Negative Predictive Value (NPV)=82 and the Area Under the Curve (AUC)=0.87.

TABLE 3A

| | |
|---|---|
| Glutaroyl-carnitine | Glycerophosphoethanolamine |
| Glycerol 2-phosphate | N-acetylglutamate |
| Nonadecanoate (19:0) | 1-stearoylglycerophosphoinositol |
| 1-myristoylglycerolphosphocholine | Creatine |
| UDP-N-acetylglucosamine | |

TABLE 3B

| | |
|---|---|
| Carnitine | 5-methylthioadenosine (MTA) |
| 2-aminoadipate | Proline |

Random Forest analysis of tissue from less aggressive, organ confined tumors (T_OC) and high aggressive, non-organ confined tumors (T_NOC) resulted in 66% accuracy. The results are presented in Table 4. A list of named biomarkers that effectively separate the genotypes are presented in Table 5.

TABLE 4
Random Forest Classification of the organ confined
tumor vs. non-organ confined cancer.

Predicted

| | | T_NOC | T_OC | class. error |
|---|---|---|---|---|
| Actual | T_NOC | 18 | 7 | 0.28 |
| | T_OC | 18 | 30 | 0.38 |

OOB error = 34%

The diagnostic parameters based on the Random Forest Analysis are that the Accuracy=66%; the Sensitivity=63%, the Specificity=72%, the Positive Predictive Value (PPV)=81%, the Negative Predictive Value (NPV)=50% and the Area Under the Curve (AUC)=0.73.

TABLE 5A

| | |
|---|---|
| Adrenate (22:4n6) | Ribitol |
| Adenosine-5-triphosphate (ATP) | Isoleucylisoleucine |
| 1-stearoylglycerol (1-monostearin) | Laurylcarnitine |
| Choline phosphate | 1-heptadecanoylglycerophospho- |
| Ethanolamine | choline |
| Caprylate (8:0) | Guanosine 5′-monophosphate (GMP) |
| 1-stearoylglycerophosphocholine | 2-aminobutyrate |
| Docosadienoate (22:2n6) | acetylcholine |

TABLE 5B

| | |
|---|---|
| Xylitol | Laurate |
| Tryptophan | Valine |
| Glycerol | Uracil |

Random Forest Analysis was also carried out to classify the tissue samples from the non-cancer tissue adjacent the high aggressive cancer tumor (N_NOC) and the non-cancer tissue adjacent the less aggressive cancer tumor (N_OC). This analysis resulted in 62% correct classification of the two tissue types. The results of the Random Forest analysis are presented in Table 6, and a list of named biomarkers that effectively separate the genotypes are presented in Tables 7A and 7B.

TABLE 6
Random Forest Classification of non-cancer tissue adjacent
to high aggressive cancer tumor (N_NOC) vs. non-cancer tissue
adjacent to less aggressive cancer tumor (N_OC).

Predicted

| | | NOC | OC | class. error |
|---|---|---|---|---|
| Actual | NOC | 15 | 10 | 0.40 |
| | OC | 17 | 29 | 0.37 |

OOB error = 38%

The diagnostic parameters based on the Random Forest Analysis are that the Accuracy=62%; the Sensitivity=63, the Specificity=60, the Positive Predictive Value (PPV)=74, the Negative Predictive Value (NPV)=47 and the Area Under the Curve (AUC)=0.71.

TABLE 7A

| | |
|---|---|
| Oleoylcarnitine | Palmitoylcarnitine |
| 3-(4-hydroxyphenyl)lactate | Taurocholenate sulfate |
| Isovalerylcarnitine | Ribitol |
| Tiglyl carnitine | Docosadienoate (22:2n6) |

TABLE 7B

| | |
|---|---|
| Hypoxanthine | Tyrosine |
| Isoleucine | Phenylalanine |
| Valine | Glycerol |
| Leucine | 5,6-dihydrouracil |
| Tryptophan | Palmitate |
| Fumarate | Kynurenine |
| S-adenosylhomocysteine (SAH) | Pantothenate |

### Example 3

### Biomarkers Useful to Rule Out Aggressive Cancer

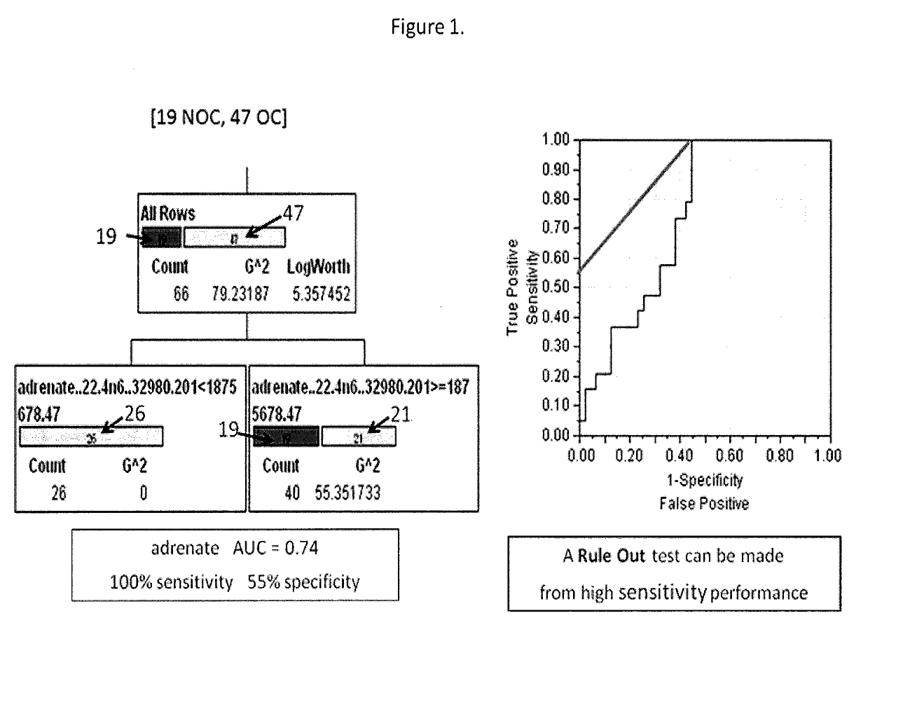

We investigated the ability of the biomarkers identified in Example 1 to rule out aggressive cancer. We selected the biomarker adrenate (22:4n6) to test this idea. The level of adrenate was measured in 19 subjects with high aggressive (i.e., NOC) cancer and 47 subjects with less aggressive (i.e., OC) cancer. The recursive partitioning analysis shows that 19 of 19 subjects with NOC cancer were classified correctly and 26 of the 47 OC subjects were classified correctly based on adrenate levels. The Sensitivity is 100% and the Specificity is 55% and the AUC is 0.74. The results are presented in FIG. 1. When these biomarkers were used to evaluate cancer aggressivity in subjects having DRE T1 or T2 and a Gleason score of 6-7, ˜40% (26/66) could be ruled out for having the aggressive form of cancer.

### Example 4

### Biomarkers Add Value to Clinical Nomograms

Currently clinicians utilize clinical parameters such as PSA, biopsy Gleason score, and DRE stage to determine PCa tumor aggressiveness. This method is not very accurate for Gleason 6-7 range. We evaluated the effects of adding metabolite biomarkers to help further stratify those with aggressive and non-aggressive disease. According to the published literature the Partin Nomogram for clinical parameters performs with an AUC of 0.68-0.73 for determining non-organ confined cancer (i.e., less aggressive cancer). We evaluated the subjects described in Example 1 using the Partin nomogram. In our dataset the Partin probabilities yielded an AUC of 0.71, consistent with the literature.

We then tested the effect of adding a pre-Rule Out Test first and then performing the Partin Nomogram on the remaining records (those not ruled out). In the dataset described in Example 1 for the Partin probabilities for subjects having Gleason 6-7 the AUC=0.65. Using the top Random Forest top hit biomarker for Gleason 6-7 subjects the AUC=0.72. For Gleason 6-7 subjects, using adrenate, the top Random Forest top hit biomarker described in Example 3 as a Rule out test first, then using the Partin probability on the remaining records the AUC increased to 0.83. These results indicate that the biomarkers identified in the instant invention can improve the performance of a currently used clinical tool for evaluating prostate cancer.

### Example 5

### DRE Urine Biomarkers

Biomarkers were identified in urine collected from subjects following a digital rectal examination (DRE) that distinguish subjects that have prostate cancer from those subjects that do not have prostate cancer. The urine was collected from the subjects (16 subjects having prostate cancer, 8 subjects not having prostate cancer) following a DRE, transferred into conical centrifuge tubes and spun in a centrifuge to separate the urine sediment from the urine liquid. The metabolites were extracted from the sediment pellet to measure the small molecules present using GC-MS and LC-MS/MS as described in the General Methods. The small molecule profiles measured in urine sediment from subjects with prostate cancer were compared with the small molecule profiles measured in urine sediment from subjects that did not have prostate cancer to identify the small molecules that are biomarkers for prostate cancer. Biomarkers were identified that correlated with the presence of cancer and were useful cancer biomarkers. The biomarkers identified that distinguish subjects having cancer from those subjects that do not have cancer are listed below in Table 8.

TABLE 8
Biomarkers

1-stearoylglycerol
3-indoxylsulfate
5-oxoproline
catechol sulfate,
glycerol 3-phosphate (G3P)
isobutyrylcarnitine
pro-hydroxy-pro
propionylcarnitine
pyruvate
uridine
threonine
3-hydroxyanthranilate
3-hydroxyhippurate
4-hydroxyhippurate
glucose
mesaconate
N-tigloylglycine
tyramine
cysteine
glycine
alanine
glutamate
sarcosine (N-methylglycine)
2-methylbutyroylcarnitine
4-acetylphenol sulfate
7-methylxanthine
arachidonate (20:4n6)
fucose
homovanillate (HVA)
indoleacetate
isovalerylcarnitine
kynurenate
leucine
N-(2-furoyl)glycine
N-acetylarginine
octanoylcarnitine
phenylacetylglycine
phenylalanine

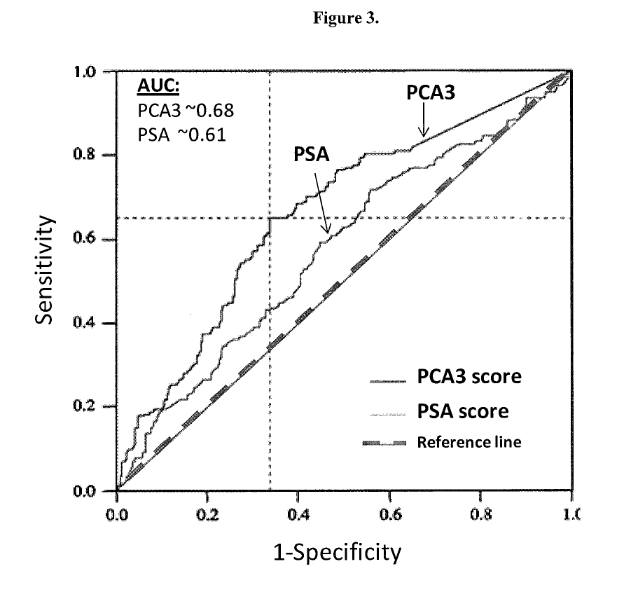

The diagnostic parameters of these biomarkers to predict prostate cancer were: Sensitivity of 81%; Specificity of 88%; PPV of 93%; NPV of 70%. The individual biomarker metabolites distinguished cancer from non-cancer with an AUC ranging from 0.73 to 0.84. Box plot graphs for representative biomarkers are presented in FIG. 3.

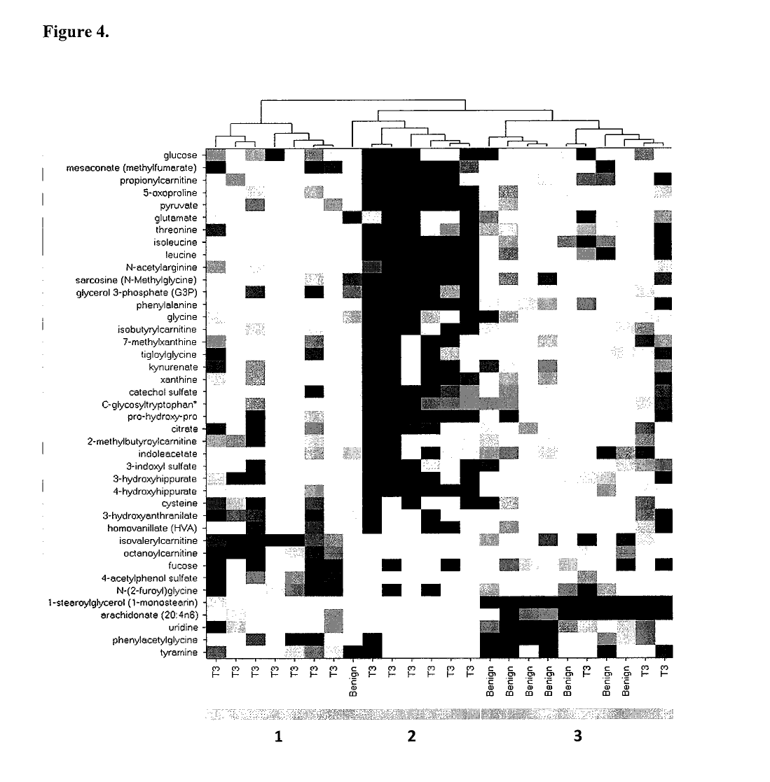

We determined that these biomarkers were useful to distinguish prostate cancer subtypes. We showed that the levels of the prostate cancer biomarkers not only produced distinct signatures that classified the subjects into prostate cancer or non-cancer groups, but also produced biomarker signatures useful to classify the prostate cancer subjects into cancer subgroups. The biomarkers and the biomarker signatures are presented in FIG. 4.

### Example 6

### Tissue Panel Biomarkers to Determine Cancer Aggressivity

Biomarkers for prostate cancer were identified in prostate tissue. The study cohort is described in Table 9. The metabolites were extracted from the prostate tissue samples that contained cancer or prostate tissue samples that did not contain cancer and the small molecules present were measured using GC-MS and LC-MS/MS as described in the general methods. To identify the prostate cancer biomarkers, the small molecule profiles measured in prostate cancer tumors were compared with the small molecule profiles measured in non-cancer prostate tissue.

TABLE 9
Study Cohort Description

| | Number of | 5 year |
|---|---|---|
| Classification | subjects | recurrence |
| Organ Confined (OC)** | 73 | 8/45 |
| Extra Capsular Extension (ECE) | 116 | 19/60 |
| (SVI negative and LN negative) | | |
| Seminal vesicle invasion positive (SVI+) | 54 | 34/43 |
| Lymph node negative (LN−) | | |
| SVI − | 7 | 6/7 |
| Lymph node positive (LN+) | | |
| SVI+ and LN+ | 25 | 19/24 |
| Total subjects | 268 | |

The biomarkers identified in prostate tissue that distinguish subjects having cancer from those subjects that do not have cancer are listed below in Table 10.

TABLE 10
Biomarkers

1-methylhistidine
1-palmitoylplasmenylethanolamine
adenosine 5′-diphosphate (ADP)
arabonate
N6-acetyllysine
N-acetylglucosamine-6-phosphate
N-acetylserine
N-formylmethionine
nicotinamide adenine dinucleotide reduced (NADH)
nicotinamide-ribonucleotide (NMN)
nicotinamide-riboside
ribulose 5-phosphate
xylulose 5-phosphate
quinate
trans-aconitate
ribose
xylulose
ethanolamine
sarcosine (N-methylglycine)
ascorbate (Vitamin C)
citrate
creatinine
inositol-1-phosphate (I1P)
kynurenine
N-acetylaspartate (NAA)
10-nonadecenoate (19:1n9)
2-palmitoylglycerophosphoethanolamine
3-(4-hydroxyphenyl)lactate
5,6-dihydrouracil
glycerol 2-phosphate
glycylvaline
lactate
N-acetylputrescine
nicotinamide-adenine-dinucleotide (NAD+)
phosphoethanolamine
putrescine
spermidine
spermine
succinylcarnitine
10-heptadecenoate (17:1n7)

Prostate cancer that is no longer confined to the prostate organ, that is, when it is not organ confined (N_OC) is considered more aggressive than prostate cancer that is confined to the prostate, that is when it is organ confined (OC). Non-organ confined prostate cancer is associated with a higher Gleason Score (GS), with detection of cancer cells in the lymph nodes (LN), with tumors that have extra-capsular extensions (ECE), and with seminal vesicle invasion (SVI). We identified biomarkers that are indicative of each of these types of aggressiveness indicators by measuring the small molecule profiles of cancer tumors with each of these aggressiveness indicators using GC-MS and LC-MS/MS as described in the general methods. The small molecule profiles obtained were compared with the small molecule profiles from non-tumor and non-aggressive cancer tumors to identify the biomarkers. The biomarkers identified in the test cohort were evaluated using a receiver operator characteristic (ROC) curve and the area under the curve (AUC) was determined for each of the aggressiveness indicators using a new cohort of subjects.

The biomarkers putrescine, lactate, 5,6-dihydrouracil, 10-nonadecenoate, NAD+, spermine, and N-acetylputrescine were useful biomarkers to indicate subjects with prostate cancer tumors that had extracapsular extensions (ECE). The AUC was 0.84.

The biomarkers putrescine, glycerol-2-phosphate, and glycylvaline were useful biomarkers to indicate subjects with prostate cancer tumors that had invaded the seminal vesicles. The AUC was 0.75.

The biomarkers phosphoethanolamine, putrescine, spermidine were useful biomarkers to indicate the subjects with prostate cancer tumors that had cancer cells detected in the lymph nodes (LN). The AUC was 0.73.

The biomarkers succinylcarnitine, 3-(4-hydroxyphenyl)lactate, 2-palmitoylglycerophosphoethanolamine, lactate, and spermidine were useful biomarkers for identifying the cancer tumors associated with a higher Gleason Score. The AUC was 0.73.

### Example 7

### Biomarkers of Prostate Cancer Recurrence

Biomarkers indicative of prostate cancer recurrence were identified that were useful to determine the individuals with prostate cancer that will recur in 5 years. Cancer recurrence is an indicator of cancer tumor aggressiveness. The levels of the biomarkers were initially measured in subjects that had prostate cancer and determined to be biomarkers for cancer aggressivity. The biomarkers were measured in an independent cohort of subjects that had been treated for prostate cancer and underwent a prostatectomy. Of this group of 61 prostate cancer subjects, the prostate cancer did not recur within 5 years in 33 subjects and prostate cancer did recur within 5 years in 28 subjects. Based on the levels of the biomarkers putrescine, lactate, 5,6-dihydrouracil, 10-nonadecenoate, NAD+, spermine, N-acetylputrescine, succinylcarnitine, 3-(4-hydroxyphenyl)lactate, 2-palmitoylglycerophosphoethanolamine, spermidine, glycerol-2-phosphate, glycylvaline, and/or phosphoethanolamine, measured in the cancer tumor tissue, the subjects were predicted to have non-aggressive cancer tumors or aggressive cancer tumors. As presented in Table 11, 25 of 28 cancer tumors that recurred within 5 years were classified as aggressive using the biomarkers while 14 of the 33 non-recurrent tumors were classified as aggressive.

TABLE 11
Cancer 5 Year Recurrence Study Cohort Description.

5 Year Recurrence (Actual)

| Predicted | Non Recurrent | Recurrent |
|---|---|---|
| Non Aggressive | 19 | 3 |
| Aggressive | 14 | 25 |

The biomarkers were useful to predict 5 year cancer recurrence. The biomarkers predicted prostate cancer recurrence in 5 years in prostate cancer subjects with a Sensitivity of 89%, Specificity of 58%, PPV of 65%, and an NPV of 86%.

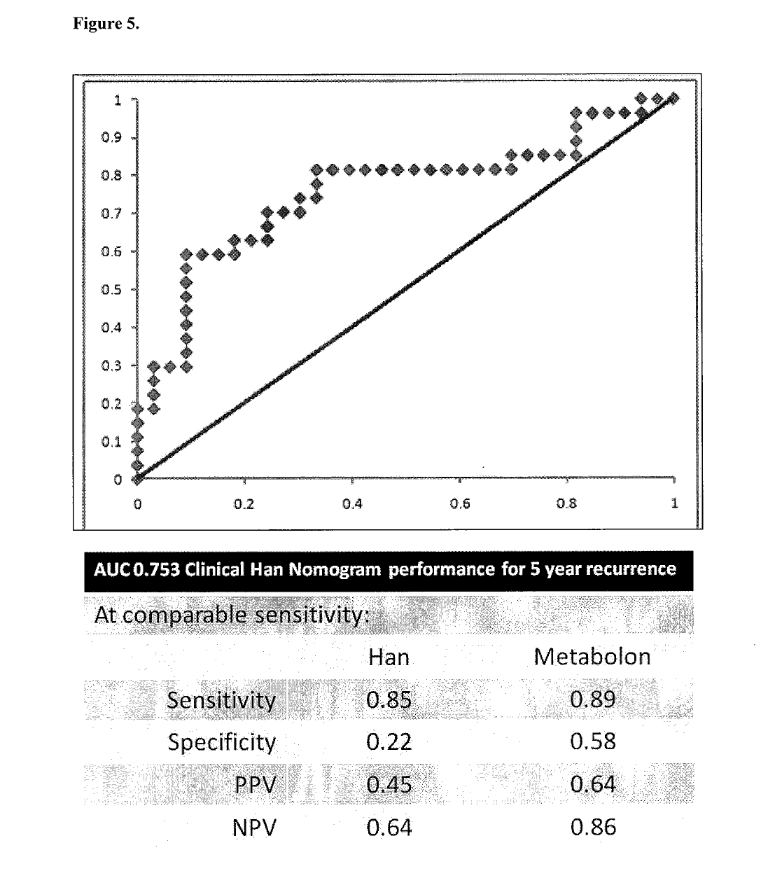

The same subjects were evaluated using the currently used clinical Han nomogram. Using the Han nomogram, 5 year cancer recurrence 23 of 27 subjects were classified correctly as recurrent. The nomogram correctly predicted non-recurrence for only 7 of 33 subjects. The results of the Han nomogram are presented in Table 12. The ROC curve for the Han nomogram is presented in FIG. 5. In contrast to the performance of the biomarkers of the instant invention, the Han nomogram had a Sensitivity of 85%, Specificity of 22%, PPV of 47% and NPV of 64%. The performance of the biomarkers in the instant invention was superior to that of the current clinical standard Han nomogram to predict the subjects with 5 year cancer recurrence.

TABLE 12
Cancer 5 Year Recurrence Predicted using Han Nomogram.

5 Year Recurrence (Actual)

| Han-Predicted: | Recurrent | Non-Recurrent |
|---|---|---|
| Recurrent | 23 | 26 |
| Non-recurrent | 4 | 7 |

The performance characteristics of the biomarkers of the instant invention and the Han nomogram are presented in Table 13.

TABLE 13
Comparison of Biomarkers with Han Nomogram
to predict cancer 5 year recurrence.

| | Han Nomogram | Biomarkers |
|---|---|---|
| Sensitivity | 0.85 | 0.89 |
| Specificity | 0.22 | 0.58 |
| PPV | 0.47 | 0.64 |
| NPV | 0.64 | 0.86 |

While the invention has been described in detail and with reference to specific embodiments thereof, it will be apparent to one skilled in the art that various changes and modifications can be made without departing from the spirit and scope of the invention.

What is claimed is:

1. A method of distinguishing low grade prostate cancer from high grade prostate cancer in a subject having prostate cancer, comprising:
analyzing a biological sample from a subject to determine the level(s) of one or more biomarkers for low grade prostate cancer and/or high grade prostate cancer in the sample, wherein the one or more biomarkers are selected from Tables 1A, 1B, 3A, 3B, 5A, 5B, 7A, 7B, 8, and/or 10 and
comparing the level(s) of the one or more biomarkers in the sample to low grade prostate cancer-positive reference levels that distinguish over high grade prostate cancer and/or to high grade prostate cancer-positive reference levels that distinguish over low grade prostate cancer in order to determine whether the subject has low grade or high grade prostate cancer.

2. The method of claim 1, wherein the one or more biomarkers are selected from Tables 1A, 1B, 5A, 5B, 7A, 7B, and/or 10.

3. The method of claim 1, wherein the biological sample is prostate tissue and the one or more biomarkers are selected from Tables 1A, 1B, 3A, 3B, 5A, 5B, 7A, 7B, 8, and/or 10.

4. The method of claim 3, wherein the one or more biomarkers are selected from Table 10.

5. The method of claim 4, wherein the one or more biomarkers are selected from putrescine, lactate, 5,6-dihydrouracil, 10-nonadecenoate, NAD+, spermine, N-acetylputrescine, succinylcarnitine, 3-(4-hydroxyphenyl)lactate, 2-palmitoylglycerophosphoethanolamine, spermidine, glycerol-2-phosphate, glycylvaline, and/or phosphoethanolamine.

6. The method of claim 5, wherein the biomarker metabolites are selected from putrescine, lactate, 5,6-dihydrouracil, 10-nonadecenoate, NAD+, spermine, and/or N-acetylputrescine.

7. The method of claim 5, wherein the biomarkers are selected from putrescine, glycerol-2-phosphate, and/or glycylvaline.

8. The method of claim 5, wherein the biomarkers are selected from phosphoethanolamine, putrescine, and/or spermidine.

9. The method of claim 5, wherein the biomarkers are selected from succinylcarnitine, 3-(4-hydroxyphenyl)lactate, 2-palmitoylglycerophosphoethanolamine, lactate, and/or spermidine.

10. The method of claim 5, wherein the biomarkers are selected from putrescine, lactate, 5,6-dihydrouracil, 10-nonadecenoate, NAD+, spermine, N-acetylputrescine, succinylcarnitine, 3-(4-hydroxyphenyl)lactate, 2-palmitoylglycerophosphoethanolamine, spermidine, glycerol-2-phosphate, glycylvaline, and/or phosphoethanolamine.

11. A method of diagnosing whether a subject has prostate cancer, comprising:
analyzing a biological sample from a subject to determine the level(s) of one or more biomarkers for prostate cancer in the sample, wherein the one or more biomarkers are selected from Tables 1A, 1B, 3A, 3B, 5A, 5B, 7A, 7B, 8, and/or 10 and
comparing the level(s) of the one or more biomarkers in the sample to prostate cancer-positive and/or prostate cancer-negative reference levels of the one or more biomarkers in order to diagnose whether the subject has prostate cancer.

12. The method of claim 11, wherein the one or more biomarkers are selected from those biomarkers in Tables 1A and/or 1B having p values of less than 0.05 and/or those biomarkers in Tables 1A and/or 1B having q values of less than 0.10.

13. The method of claim 11, wherein the one or more biomarkers are selected from Tables 1A, 1B, 3A, 3B, and 8.

14. The method of claim 11, wherein the method comprises analyzing the biological sample to determine the level of two or more biomarkers selected from Tables 1A, 1B, 3A, 3B, 5A, 5B, 7A, 7B, 8, and/or 10.

15. The method of claim 11, wherein the method comprises analyzing the biological sample to determine the level of three or more biomarkers selected from Tables 1A, 1B, 3A, 3B, 5A, 5B, 7A, 7B, 8, and/or 10.

16. The method of claim 11, wherein the method comprises analyzing the biological sample to determine the level of four or more biomarkers selected from Tables 1A, 1B, 3A, 3B, 5A, 5B, 7A, 7B, 8, and/or 10.

17. The method of claim 11, wherein the method comprises analyzing the biological sample to determine the level of five or more biomarkers selected from Tables 1A, 1B, 3A, 3B, 5A, 5B, 7A, 7B, 8, and/or 10.

18. The method of claim 11, wherein the method comprises analyzing the biological sample to determine the level of ten or more biomarkers selected from Tables 1A, 1B, 3A, 3B, 5A, 5B, 7A, 7B, 8, and/or 10.

19. The method of claim 11, wherein the method comprises analyzing the biological sample to determine the level of fifteen or more biomarkers selected from Tables 1A, 1B, 3A, 3B, 5A, 5B, 7A, 7B, 8, and/or 10.

20. The method of claim 11, wherein the biological sample is prostate tissue and the one or more biomarkers are selected from Tables 1A, 1B, 3A, 3B, 5A, 5B, 7A, 7B, 8, and/or 10.

21. The method of claim 11, wherein the biological sample is prostate tissue and the one or more biomarkers are selected from Tables 1A, 1B, 3A, 3B, 5A, 5B, 7A, 7B, and/or 10.

22. The method of claim 11, wherein the biological sample is urine and the one or more biomarkers are selected from Tables 1A, 1B, 3A, 3B, 5A, 5B, 7A, 7B, 8, and/or 10.

23. The method of claim 22, wherein the one or more biomarkers are selected from Table 8.

24. The method of claim 23, wherein the biological sample is a DRE urine sample.

25. The method of claim 11, wherein the sample is analyzed using one or more techniques selected from the group consisting of mass spectrometry, ELISA, and antibody linkage.

26. A method of determining whether a subject is predisposed to developing prostate cancer, comprising:
analyzing a biological sample from a subject to determine the level(s) of one or more biomarkers for prostate cancer in the sample, wherein the one or more biomarkers are selected from Tables 1A, 1B, 3A, 3B, 5A, 5B, 7A, 7B, 8 and/or 10; and
comparing the level(s) of the one or more biomarkers in the sample to prostate cancer-positive and/or prostate cancer-negative reference levels of the one or more biomarkers in order to determine whether the subject is predisposed to developing prostate cancer.

27. A method of monitoring progression/regression of prostate cancer in a subject comprising:
analyzing a first biological sample from a subject to determine the level(s) of one or more biomarkers for prostate cancer in the sample, wherein the one or more biomarkers are selected from Tables 1A, 1B, 3A, 3B, 5A, 5B, 7A, 7B, 8 and/or 10 and the first sample is obtained from the subject at a first time point;
analyzing a second biological sample from a subject to determine the level(s) of the one or more biomarkers, wherein the second sample is obtained from the subject at a second time point; and
comparing the level(s) of one or more biomarkers in the first sample to the level(s) of the one or more biomarkers in the second sample in order to monitor the progression/regression of prostate cancer in the subject.

28. The method of claim 22, wherein the method further comprises comparing the level(s) of one or more biomarkers in the first sample, the level(s) of one or more biomarkers in the second sample, and/or the results of the comparison of the level(s) of the one or more biomarkers in the first and second samples to prostate cancer-positive and/or prostate cancer-negative reference levels of the one or more biomarkers.

29. A method of assessing the efficacy of a composition for treating prostate cancer comprising:
analyzing, from a subject having prostate cancer and currently or previously being treated with a composition, a biological sample to determine the level(s) of one or more biomarkers for prostate cancer selected from Tables 1A, 1B, 3A, 3B, 5A, 5B, 7A, 7B, 8 and/or 10; and
comparing the level(s) of the one or more biomarkers in the sample to (a) levels of the one or more biomarkers in a previously-taken biological sample from the subject, wherein the previously-taken biological sample was obtained from the subject before being treated with the composition, (b) prostate cancer-positive reference levels of the one or more biomarkers, and/or (c) prostate cancer-negative reference levels of the one or more biomarkers.

30. A method for assessing the efficacy of a composition in treating prostate cancer, comprising:
analyzing a first biological sample from a subject to determine the level(s) of one or more biomarkers for prostate cancer selected from Tables 1A, 1B, 3A, 3B, 5A, 5B, 7A, 7B, 8 and/or 10, the first sample obtained from the subject at a first time point;
administering the composition to the subject;
analyzing a second biological sample from the subject to determine the level(s) of the one or more biomarkers, the second sample obtained from the subject at a second time point after administration of the composition;
comparing the level(s) of one or more biomarkers in the first sample to the level(s) of the one or more biomarkers in the second sample in order to assess the efficacy of the composition for treating prostate cancer.

31. A method of assessing the relative efficacy of two or more compositions for treating prostate cancer comprising:
analyzing, from a first subject having prostate cancer and currently or previously being treated with a first composition, a first biological sample to determine the level(s) of one or more biomarkers selected from Tables 1A, 1B, 3A, 3B, 5A, 5B, 7A, 7B, 8 and/or 10;
analyzing, from a second subject having prostate cancer and currently or previously being treated with a second composition, a second biological sample to determine the level(s) of the one or more biomarkers; and
comparing the level(s) of one or more biomarkers in the first sample to the level(s) of the one or more biomarkers in the second sample in order to assess the relative efficacy of the first and second compositions for treating prostate cancer.

32. A method for screening a composition for activity in modulating one or more biomarkers of prostate cancer, comprising:
contacting one or more cells with a composition;
analyzing at least a portion of the one or more cells or a biological sample associated with the cells to determine the level(s) of one or more biomarkers of prostate cancer selected from Tables 1A, 1B, 3A, 3B, 5A, 5B, 7A, 7B, 8 and/or 10; and
comparing the level(s) of the one or more biomarkers with predetermined standard levels for the biomarkers to determine whether the composition modulated the level(s) of the one or more biomarkers.

33. The method of claim 32, wherein the predetermined standard levels for the biomarkers are level(s) of the one or more biomarkers in the one or more cells in the absence of the composition.

34. The method of claim 32, wherein the predetermined standard levels for the biomarkers are level(s) of the one or more biomarkers in one or more control cells not contacted with the composition.

35. The method of claim 32, wherein the method is conducted in vivo.

36. The method of claim 32, wherein the method is conducted in vitro.

37. A method for identifying a potential drug target for prostate cancer comprising:
identifying one or more biochemical pathways associated with one or more biomarkers for prostate cancer selected from Tables 1A, 1B, 3A, 3B, 5A, 5B, 7A, 7B, 8 and/or 10; and
identifying a protein affecting at least one of the one or more identified biochemical pathways, the protein being a potential drug target for prostate cancer.

38. A method for treating a subject having prostate cancer comprising administering to the subject an effective amount of one or more biomarkers selected from Tables 1A, 1B, 3A, 3B, 5A, 5B, 7A, 7B, 8 and/or 10 that are decreased in prostate cancer.
